(19) United States
(12) Patent Application Publication
(10) Pub. No.: US 2026/0041391 A1
(43) Pub. Date: Feb. 12, 2026

(54) MACHINE LEARNING ENABLED LONGITUDINAL ANALYSIS OF POSITRON EMISSION TOMOGRAPHY AND COMPUTED TOMOGRAPHY SCANS FOR ASSESSMENT OF DISEASE PROGRESSION AND TREATMENT RESPONSE

(71) Applicant: Genentech, Inc., South San Francisco, CA (US)

(72) Inventors: Xiaoyong WANG, South San Francisco, CA (US); Nils Gustav Thomas BENGTSSON, San Francisco, CA (US); Richard Alan Duray CARANO, San Ramon, CA (US); Alexander James Stephen CHAMPION DE CRESPIGNY, Redwood City, CA (US); Mohamed Skander JEMAA, San Francisco, CA (US)

(21) Appl. No.: 19/364,664

(22) Filed: Oct. 21, 2025

Related U.S. Application Data
(63) Continuation of application No. PCT/US2024/025448, filed on Apr. 19, 2024.
(60) Provisional application No. 63/497,660, filed on Apr. 21, 2023.

(51) Int. Cl. A61B 6/00; A61B 6/03; G06T 7/00; G06T 7/11; G06T 7/33; G16H 20/40

(52) U.S. Cl. CPC A61B 6/5217; A61B 6/03; A61B 6/037; A61B 6/5235; G06T 7/0016; G06T 7/11; G06T 7/337; G16H 20/40; G06T 2207/10081; G06T 2207/10104; G06T 2207/30096

(57) ABSTRACT

A method may include determining, based on a first positron emission tomography (PET) scan and a first computed tomography (CT) scan from a first timepoint, a first tumor mask corresponding to a first lesion present in the first PET scan and the first CT scan. A second tumor mask corresponding to a second lesion present in the second PET scan and the second CT scan may be determined based on the second PET scan and the second CT scan from a second timepoint. A longitudinal segmentation model may be applied to update, based on the first PET scan, the first CT scan, the second PET scan, and the second CT scan, each of the first tumor mask and the second tumor mask. A response to a treatment for a disease may be determined based on at least one of the first updated tumor mask and the second updated tumor mask. Related systems and computer program products are also provided.

### CROSS REFERENCE TO RELATED APPLICATION

This application claims priority to U.S. Provisional Application 63/497,660, entitled “MACHINE LEARNING ENABLED LONGITUDINAL ANALYSIS OF POSITRON EMISSION TOMOGRAPHY AND COMPUTED TOMOGRAPHY SCANS FOR ASSESSMENT OF DISEASE PROGRESSION AND TREATMENT RESPONSE” and filed on Apr. 21, 2023, the disclosure of which is incorporated herein by reference in its entirety.

### TECHNICAL FIELD

The subject matter described herein relates generally to machine learning and more specifically to machine learning based technique for assessing disease progression and treatment response based on positron emission tomography (PET) and computed tomography (CT) scans.

### INTRODUCTION

Medical imaging refers to techniques and processes for obtaining data characterizing a subject's internal anatomy and pathophysiology including, for example, images created by the detection of radiation either passing through the body (e.g. x-rays) or emitted by administered radiopharmaceuticals (e.g. gamma rays from intravenously administered radioactive tracers). By revealing internal anatomical structures obscured by other tissues such as skin, subcutaneous fat, and bones, medical imagining is integral to numerous medical diagnosis and/or treatments. Examples of medical imaging modalities include 2-dimensional imaging such as x-ray plain films, bone scintigraphy, and thermography. Examples of 3-dimensional imaging modalities include magnetic resonance imaging (MRI), computed tomography (CT), cardiac sestamibi scanning, and positron emission tomography (PET).

### SUMMARY

Systems, methods, and articles of manufacture, including computer program products, are provided for machine learning enabled longitudinal analysis of positron emission tomography (PET) and computed tomography (CT) scans for assessing disease progression and treatment response. Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

In one aspect, there is a provided a system for machine learning enabled longitudinal analysis of positron emission tomography (PET) and computed tomography (CT) scans for assessing disease progression and treatment response. The system may include at least one processor and at least one memory. The at least one memory may include program code that provides operations when executed by the at least one processor. The operations may include: determining, based at least on a first positron emission tomography (PET) scan and a first computed tomography (CT) scan from a first timepoint, a first tumor mask corresponding to a first lesion present in the first PET scan and the first CT scan; determining, based at least on a second PET scan and a second CT scan from a second timepoint, a second tumor mask corresponding to a second lesion present in the second PET scan and the second CT scan; applying a longitudinal segmentation model to update, based at least on the first PET scan, the first CT scan, the second PET scan, and the second CT scan, each of the first tumor mask and the second tumor mask; and determining, based on at least one of the first updated tumor mask and the second updated tumor mask, a response to a treatment for a disease.

In another aspect, there is provided a method for machine learning enabled longitudinal analysis of positron emission tomography (PET) and computed tomography (CT) scans for assessing disease progression and treatment response. The method may include: determining, based at least on a first positron emission tomography (PET) scan and a first computed tomography (CT) scan from a first timepoint, a first tumor mask corresponding to a first lesion present in the first PET scan and the first CT scan; determining, based at least on a second PET scan and a second CT scan from a second timepoint, a second tumor mask corresponding to a second lesion present in the second PET scan and the second CT scan; applying a longitudinal segmentation model to update, based at least on the first PET scan, the first CT scan, the second PET scan, and the second CT scan, each of the first tumor mask and the second tumor mask; and determining, based on at least one of the first updated tumor mask and the second updated tumor mask, a response to a treatment for a disease.

In another aspect, there is provided a computer program product for machine learning enabled longitudinal analysis of positron emission tomography (PET) and computed tomography (CT) scans for assessing disease progression and treatment response. The computer program product may include a non-transitory computer readable medium storing instructions that cause operations when executed by at least one data processor. The operations may include: determining, based at least on a first positron emission tomography (PET) scan and a first computed tomography (CT) scan from a first timepoint, a first tumor mask corresponding to a first lesion present in the first PET scan and the first CT scan; determining, based at least on a second PET scan and a second CT scan from a second timepoint, a second tumor mask corresponding to a second lesion present in the second PET scan and the second CT scan; applying a longitudinal segmentation model to update, based at least on the first PET scan, the first CT scan, the second PET scan, and the second CT scan, each of the first tumor mask and the second tumor mask; and determining, based on at least one of the first updated tumor mask and the second updated tumor mask, a response to a treatment for a disease.

In some variations of the methods, systems, and non-transitory computer readable media, one or more of the following features can optionally be included in any feasible combination.

In some variations, the method may determine, based at least on a first positron emission tomography (PET) scan and a first computed tomography (CT) scan from a first timepoint, a first tumor mask corresponding to a first lesion present in the first PET scan and the first CT scan; determine, based at least on a second PET scan and a second CT scan from a second timepoint, a second tumor mask corresponding to a second lesion present in the second PET scan and the second CT scan; apply a longitudinal segmentation model to update, based at least on the first PET scan, the first CT scan, the second PET scan, and the second CT scan, each of the first tumor mask and the second tumor mask; and determine, based on at least one of the first updated tumor mask and the second updated tumor mask, a response to a treatment for a disease.

In some variations, the first tumor mask may identify a first plurality of pixels in each of the first PET scan and the first CT scan depicting the first lesion, and wherein the second tumor mask identifies a plurality of pixels from the second PET scan and the second CT scan depicting the second lesion.

In some variations, the method may register the first CT scan, the first PET scan, the second CT scan, and the second PET scan in order to align the first CT scan and the first PET scan with the second CT scan and the second PET scan.

In some variations, the method may identify, based at least on the first updated tumor mask and the second updated tumor mask, the second lesion as a new lesion; and in response to the second lesion being identified as the new lesion, may determine the response to the treatment as progressive disease (PMD).

In some variations, the method may determine, based at least on the first updated tumor mask and the second updated tumor mask, a distance between the first lesion and the second lesion; identify the second lesion as the new lesion based at least on the distance between the first lesion and the second lesion satisfying one or more thresholds; and identify the second lesion as a same lesion as the first lesion based at least on the distance between the first lesion and the second lesion failing to satisfy the one or more thresholds.

In some variations, the method may in response to determining that the first lesion and the second lesion are a same lesion, may determine the response to the treatment for the disease based at least on a change in metabolic activity exhibited by the lesion between the first timepoint and the second timepoint.

In some variations, the change in metabolic activity between the first timepoint and the second timepoint may be determined by at least determining, based at least on the first updated tumor mask and the first PET scan, a first level of metabolic activity exhibited by the lesion at the first timepoint, determining, based at least on the second updated tumor mask and the second PET scan, a second level of metabolic activity exhibited by the lesion at the second timepoint, and determining, based at least on the first level of metabolic activity and the second level of metabolic activity, the change in metabolic activity between the first timepoint and the second timepoint.

In some variations, the response to the treatment is determined as progressive metabolic disease (PMD) based at least on the change in metabolic activity between the first timepoint and the second timepoint satisfying a first threshold.

In some variations, the response to the treatment may be determined as no metabolic response (NMR) based at least on the change in metabolic activity between the first timepoint and the second

In some variations, the response to the treatment may be determined as partial metabolic response (PMR) based at least on the change in metabolic activity between the first timepoint and the second timepoint failing to satisfy the first threshold and the second threshold.

In some variations, the first level of metabolic activity may correspond to a first standardized uptake value (SUV) and the second level of metabolic activity corresponds to a second standardized uptake (SUV) value.

In some variations, each of the first level of metabolic activity and the second level of metabolic activity may correspond to a maximum, a minimum, a median, a mean, or a mode level of metabolic activity exhibited by the lesion at a corresponding timepoint.

In some variations, the first CT scan and the first PET scan may be performed prior to the treatment for the disease, and the second CT scan and the second PET scan may be performed subsequent to the treatment for the disease.

In some variations, the method may determine, based at least on the first updated tumor mask and the second updated tumor mask, a change in tumor volume; and may determine, based at least on the change in tumor volume, the response to the treatment for the disease.

In some variations, the method may determine, based at least on the first updated tumor mask and the second updated tumor mask, a variance in a first change in metabolic activity and/or a second change in tumor volume between the first timepoint and the second timepoint across different lesions; and may determine the response to the treatment based at least on the variance in the change in metabolic activity and/or tumor volume between the first timepoint and the second timepoint exhibited by the different lesions.

In some variations, the method may determine, based at least on the first updated tumor mask and the second updated tumor mask, a progression of the disease.

In some variations, the first tumor mask may be determined by applying a segmentation model to the first PET scan and the first CT scan, and the second tumor mask may be determined by applying the segmentation model to the second PET scan and the second CT scan.

In some variations, longitudinal segmentation model may be an artificial neural network or a vision transformer.

In some variations, each of the first CT scan, the first PET scan, the second CT scan, and the second PET scan may be a three-dimensional volume comprising a plurality of two-dimensional patches.

In some variations, each pixel in the first PET scan and the second PET scan may be associated with an intensity value corresponding to a level of metabolic activity.

In some variations, each pixel in the first CT scan and the second CT scan may be associated with an intensity value corresponding to a tissue density or X-ray attenuation.

In some variations, the method may train the longitudinal segmentation model to update two or more tumor masks, each tumor mask of the two or more tumor masks being generated from a positron emission tomography (PET) scan and a computed tomography (CT) scan from a single timepoint.

In some variations, the response to the treatment for the disease may be complete metabolic response (CMR) or non-complete metabolic response (non-CMR).

In some variations, the response to the treatment for the disease may be responder or non-responder.

In some variations, the response to the treatment for the disease may be complete metabolic response (CMR), partial metabolic response (PMR), no metabolic response (NMR), or progressive metabolic disease (PMD).

In some variations, the method may extract, from the first PET scan and the first CT scan, a first patch including the first lesion associated with first tumor mask; extract, from the second PET scan and the first CT scan, a second patch including the second lesion associated with the second tumor mask; and apply the longitudinal segmentation model to the first patch and the second patch in order to update each of the first tumor mask and the second tumor mask.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to fluorodeoxyglucose avid (FDG-avid) cancers such as some types of non-Hodgkin lymphoma (NHL), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a machine learning based medical imaging analysis system, in accordance with some example embodiments;

FIG. 2 depicts a schematic diagram illustrating an example of a process for machine learning enabled longitudinal analysis of positron emission tomography (PET) and computed tomography (CT) scans, in accordance with some example embodiments;

FIG. 3 depicts a flowchart illustrating an example of a process for machine learning enabled longitudinal analysis of positron emission tomography (PET) and computed tomography (CT) scans, in accordance with some example embodiments;

FIG. 4 depicts a flowchart illustrating another example of a process for machine learning enabled longitudinal analysis of positron emission tomography (PET) and computed tomography (CT) scans, in accordance with some example embodiments;

FIG. 5 depicts a flowchart illustrating another example of a process for machine learning enabled longitudinal analysis of positron emission tomography (PET) and computed tomography (CT) scans, in accordance with some example embodiments;

FIG. 6 depicts a comparison of accuracies for complete metabolic response (CMR) assessment and objective response (OR) assessment made by machine learning enabled longitudinal analysis and inter-radiologist agreement across different clinical datasets, in accordance with some example embodiment;

FIG. 7 depicts a performance comparison for machine learning enabled longitudinal analysis and expert radiologists, in accordance with some example embodiments;

FIG. 8A depicts a comparison of the accuracies for complete metabolic response (CMR) assessment and objective response (OR) assessment made by machine learning enabled longitudinal analysis and inter-radiologist agreement, in accordance with some example embodiments;

FIG. 8B depicts a comparison of the accuracy of the response assessment made machine learning enabled longitudinal analysis and inter-radiologist agreement, in accordance with some example embodiments;

FIG. 8C depicts a comparison of F1-scores for machine learning enabled longitudinal analysis and radiologist analysis, in accordance with some example embodiments;

FIG. 8D depicts an evaluation of the progression free survival (PFS) by end of treatment assessment made by machine learning enabled longitudinal analysis and of the progression free survival (PFS) by end of treatment made by an expert radiologist panel across different clinical datasets, in accordance with some example embodiments;

FIG. 8E depicts a comparison of the accuracy of the complete metabolic response (CMR) assessment, objective response (OR) assessment, and four-category assessment made by machine learning enabled longitudinal analysis and by expert radiologists, in accordance with some example embodiments;

FIG. 8F depicts a comparison of the accuracy of machine learning enabled longitudinal analysis and expert radiologist analysis, in accordance with some example embodiments;

FIG. 8G depicts a comparison the overall (OS) by end of treatment assessment made by machine learning enabled longitudinal analysis and adjudicated responses across different clinical datasets, in accordance with some example embodiments;

FIG. 9 depicts overall survival (OS) by end of treatment and early discontinuation as determined by machine learning enabled longitudinal analysis across various clinical datasets, in accordance with some example embodiments;

FIG. 10 depicts a comparison of Deauville Score (DS) assessment made by machine learning enabled longitudinal analysis and adjudicated responses across different clinical datasets, in accordance with some example embodiments.

FIG. 11 depicts examples of true positives, true negatives, false positives and false negatives.

FIG. 12 depicts a block diagram illustrating an example of a computing system, in accordance with some example embodiments.

Materials,” the disclosure of which is incorporated herein by reference in its entirety.

When practical, similar reference numbers denote similar structures, features, or elements.

### DETAILED DESCRIPTION

Various modalities of medical imaging may be applied to obtain data characterizing a subject's internal anatomy as well as pathophysiology. Computed tomography (CT) is an example of a three-dimensional imaging modality in which a series of X-rays are captured to create cross-sectional images (e.g., patches, slices, and/or the like) of the bones, blood vessels, and soft tissues inside the body. A computed tomography scan may be a three-dimensional volume formed by a series of two-dimensional images in which each pixel is associated with an intensity value indicative of a tissue density or x-ray attenuation at the corresponding location in the subject's body. Another example of a three-dimensional imaging modality is positron emission tomography (PET), which captures radioactivity signals indicative of cellular metabolic activities inside the subject's body. A positron emission tomography scan may be a three-dimensional volume formed by a series of two-dimension images in which each pixel is associated with an intensity value indicative of the level of cellular metabolic activity (e.g., glucose uptake) at the corresponding location in the subject's body. In some cases, a single gantry incorporating a positron emission tomography (PET) scanner and a computed tomography (CT) scanner may be capable of acquiring positron emission tomography (PET) scans and computed tomography (CT) scans during a same session. The resulting positron emission tomography (PET) scan and computed tomography (CT) scan may be combined into a single superposed (e.g., co-registered) image (e.g., a PET-CT scan) in which the spatial distribution of metabolic activities depicted in the positron emission tomography (PET) scan is aligned with the anatomical structures depicted in the computed tomography (CT) scan.

In some example embodiments, an analysis controller may perform longitudinal analysis of positron emission tomography (PET) scans and computed tomography (CT) scans for assessing disease progression and treatment response. In some cases, the analysis controller may apply a longitudinal segmentation model trained to update two or more individual tumor masks generated from positron emission tomography (PET) scans and computed tomography (CT) scans from individual timepoints. For example, the longitudinal segmentation model may ingest a first tumor mask determined based on a first positron emission tomography (PET) scan and a first computed (CT) scan from a first timepoint, and corresponding to a first lesion present in the first positron emission tomography (PET) scan and a first computed (CT) scan. Furthermore, the longitudinal segmentation model may ingest a second tumor mask determined based on a second positron emission tomography (PET) scan and a second computed tomography (CT) scan from a second timepoint, and corresponding to a second lesion present in the second positron emission tomography (PET) scan and the second computed tomography (CT) scan. The longitudinal segmentation model may update each of the first tumor mask and the second tumor mask based on the first positron emission tomography (PET) scan, the first computed tomography (CT) scan, the second positron emission tomography (PET) scan, and the second computed tomography (CT) scan. In doing so, the longitudinal segmentation model may refine the first tumor mask and the second tumor mask to reduce false positives in which one or more pixels that are not a part of a lesion are incorrectly identified as such.

In some example embodiments, the analysis controller may determine, based at least on the updated tumor masks generated by the longitudinal segmentation model, a response to a treatment for a disease (e.g., complete metabolic response (CMR), objective response (OR), four-category assessment, and/or the like). Alternatively and/or additionally, the analysis controller may determine, based at least on the updated tumor masks generated by the longitudinal segmentation model, a progression of the disease. For example, in some cases, the analysis controller may determine, based at least on the first updated tumor mask and the second updated tumor mask, the response to a treatment for a disease associated with the first lesion and the second lesion. In some cases, the analysis controller may determine, based at least on the first updated tumor mask and the second updated tumor mask, the response to the treatment for the disease as progressive metabolic disease (PMD), no metabolic response (NMR), partial metabolic response (PMR), or complete metabolic response (CMR). Alternatively, in some cases, the analysis controller may determine, based at least on the first updated tumor mask and the second updated tumor mask, the response to the treatment for the disease as complete metabolic response (CMR) or non-complete metabolic response (non-CMR).

In some example embodiments, the analysis controller may identify, based at least on the first updated tumor mask and the second updated tumor mask, the second lesion present in the second positron emission tomography (PET) scan and the second computed tomography (CT) scan as a new lesion that is not present in the first positron emission tomography (PET) scan and the first computed tomography (CT) scan. For example, in some cases, the analysis controller may determine that the second lesion is a new lesion if the distance (e.g., a minimal distance, an average distance, and/or the like) between the first lesion and the second lesion satisfies one or more thresholds (e.g., a minimal distance exceeding 10 millimeters). Otherwise, if the distance between the first lesion and the second lesion fails to satisfy the one or more thresholds, the analysis controller may determine that the first lesion and the second lesion are the same lesion. Accordingly, in instances where the second lesion is identified as a new lesion, the analysis controller may determine that the response to the treatment for the disease as progressive metabolic response (PMR). In instances where the second lesion is identified as a same lesion as the first lesion, the analysis controller may further determine the response to the treatment for the disease based on a change in the level of metabolic activity exhibited by the lesion between the first timepoint and the second timepoint. For instance, the analysis controller may determine the response to the treatment for the disease as progressive metabolic disease (PMD) where the change in metabolic activity between the first timepoint and the second timepoint satisfies a first threshold, no metabolic response (NMR) where the change in metabolic activity satisfies a second threshold but not the first threshold, and partial metabolic response (PMR) where the change in metabolic activity fails to satisfy the first threshold as well as the second threshold.

In some example embodiments, the analysis controller may determine, based at least on the first updated tumor mask and the second updated tumor mask, the change in the level of metabolic activity exhibited by the lesion between the first timepoint and the second timepoint. The change in the level of metabolic activity may correspond to a change in a variety of metrics derived based on the updated tumor masks. Examples of such metrics standard uptake values (e.g., maximum standard uptake value, minimum standard uptake value, median standard uptake value, mean standard uptake value, mode standard uptake value, and/or the like) and lesion size. For example, in some cases, the change in the level of metabolic activity exhibited by the lesion between the first timepoint and the second timepoint may correspond to a difference between the first maximum level of metabolic activity at the first timepoint and the second maximum level of metabolic activity at the second timepoint. Accordingly, in some cases, the analysis controller may determine, based at least on the first updated tumor mask, a first maximum level of metabolic activity (e.g., a first maximum standard uptake value (SUVₘₐₓ)) at the first timepoint. Moreover, the analysis controller may determine, based at least on the second updated tumor mask, a second maximum level of metabolic activity (e.g., a second maximum standard uptake value (SUVₘₐₓ)) at the second timepoint. As noted, in some cases where the analysis controller failed to identify a new lesion, the analysis controller may determine the response to the treatment based on whether the change in the level of metabolic activity exhibited by the lesion between the first timepoint and the second timepoint satisfies one or more thresholds.

In some example embodiments, the analysis controller may also determine, based at least on the first updated tumor mask and the second updated tumor mask, a change in tumor volume (e.g., total metabolic tumor volume (TMTV)) between the first timepoint and the second timepoint. In some cases, the response to the treatment for the disease may be determined based on the change in tumor volume between the first timepoint and the second timepoint. Alternatively and/or additionally, the analysis controller may determine, based at least on the first updated tumor mask and the second updated tumor mask, a variance in the change in metabolic activity and/or tumor volume between the first timepoint and the second timepoint across different lesions. In some cases, the analysis controller may determine the response to the treatment for the disease further based on the variance in the change in metabolic activity and/or tumor volume between the first timepoint and the second timepoint across different lesions.

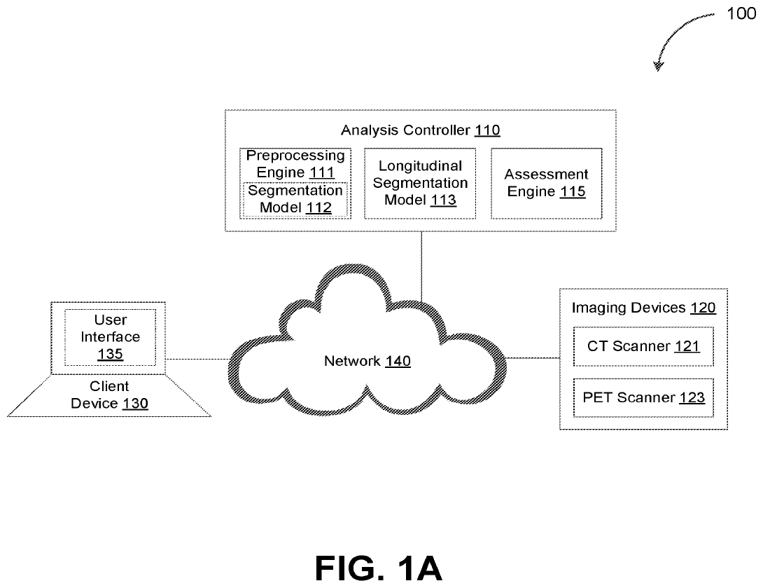

FIG. 1 depicts a system diagram illustrating an example of a machine learning based medical imaging analysis system 100, in accordance with some example embodiments. Referring to FIG. 1, the machine learning based medical imaging analysis system 100 may include an analysis controller 110, one or more imaging devices 120, and a client device 130. As shown in FIG. 1, the analysis controller 110, the one or more imaging devices 120, and the client device 130 may be communicatively coupled via a network 140. The one or more imaging devices 120 may include, for example, a computed tomography (CT) scanner 121 and a positron emission tomography (PET) scanner 123. The client device 130 may be a processor-based device including, for example, a smartphone, a tablet computer, a wearable apparatus, a virtual assistant, an Internet-of-Things (IoT) appliance, and/or the like. The network 140 may be a wired network and/or a wireless network including, for example, a wide area network (WAN), a local area network (LAN), a virtual local area network (VLAN), a public land mobile network (PLMN), the Internet, and/or the like.

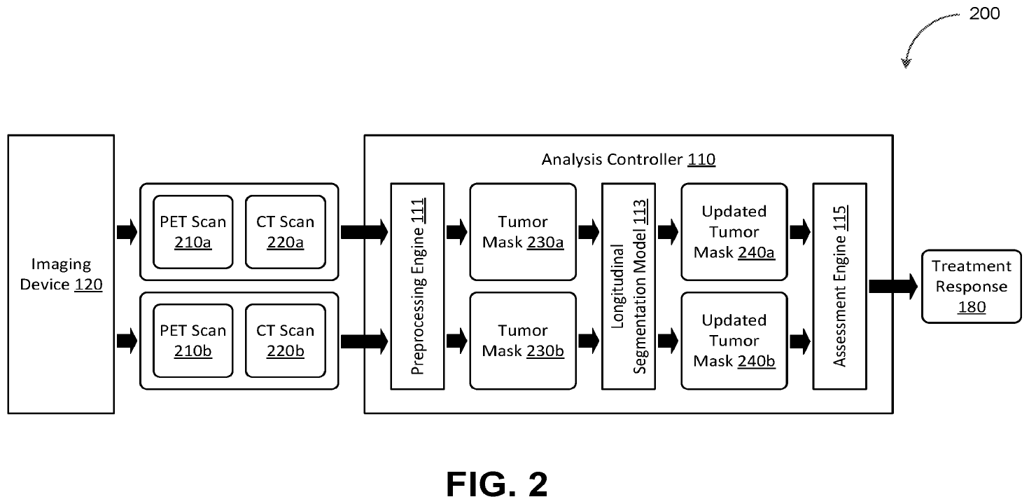

In some example embodiments, the analysis controller 110 may perform longitudinal analysis of positron emission tomography (PET) scans and computed tomography (CT) scans generated by the one or more imaging devices 120 (e.g., the computed tomography scanner 121, the positron emission tomography scanner (PET) scanner 123, and/or the like). In some cases, the analysis controller 110 may apply a longitudinal segmentation model 113, which may be trained to update two or more individual tumor masks generated from positron emission tomography (PET) scans and computed tomography (CT) scans from individual timepoints. To further illustrate, FIG. 2 depicts a schematic diagram illustrating an example of a process 200 for machine learning enabled longitudinal analysis of positron emission tomography (PET) and computed tomography (CT) scans, in accordance with some example embodiments.

Referring to FIG. 2, the analysis controller 110 may receive, from the one or more imaging devices 120, a first positron emission tomography (PET) scan 210a and a first computed tomography (CT) scan 220a from a first timepoint. Furthermore, the analysis controller 110 may also receive, from the one or more imaging devices 120, a second positron emission tomography (PET) scan 210b and a second computed tomography (CT) scan 220b from a second timepoint. In some cases, the analysis controller 110 may include a preprocessing controller 110. In the example shown in FIG. 2, the preprocessing controller 110 may preprocess the first positron emission tomography (PET) scan 210a and the first computed tomography (CT) scan 220a to generate a first tumor mask 230a. Moreover, the preprocessing controller 110 may also preprocess the second positron emission tomography (PET) scan 210b and the second computed tomography (CT) scan 220b to generate a second tumor mask 230b.

In some example embodiments, the preprocessing may include registration to align the first positron emission tomography (PET) scan 210a and the first computed tomography (CT) scan 220a with the second positron emission tomography (PET) scan 210b and the second computed tomography (CT) scan 220b. For example, in some cases, the preprocessing engine 111 may perform an affine and block matching registration based on the first computed tomography (CT) scan 220a from the first timepoint and the second computed tomography (CT) scan 220b from the second timepoint. Accordingly, the first positron emission tomography (PET) scan 210a may be superimposed (or co-registered) with the first computed tomography (CT) scan 220a while the second positron emission tomography (PET) scan 210b may be superimposed (or co-registered) with the second computed tomography (CT) scan 220b. Furthermore, the first positron emission tomography (PET) scan 210a and the first computed tomography (CT) scan 220a from the first timepoint may be superimposed (or co-registered) with the second positron emission tomography (PET) scan 210b and the second computed tomography (CT) scan 220b. In doing so, the preprocessing engine 111 may map a first pixel in the first positron emission tomography (PET) scan 210a to a second pixel in the first computed tomography (CT) scan 220a, a third pixel in the second positron emission tomography (PET) scan 210b, and a fourth pixel in the second computed tomography (CT) scan 220b.

In some example embodiments, the preprocessing may also include partitioning the first positron emission tomography (PET) scan 210a, the first computed tomography (CT) scan 220a, the second positron emission tomography (PET) scan 210b, and the second computed tomography (CT) scan 220b into two or more regions. For instance, in some cases, the preprocessing engine 111 may partition, based on one or more anatomical landmarks, the first positron emission tomography (PET) scan 210a, the first computed tomography (CT) scan 220a, the second positron emission tomography (PET) scan 210b, and the second computed tomography (CT) scan 220b into two or more anatomical regions. Examples of anatomical regions include a head and neck region, a chest region, and an abdomen and pelvis region.

In some example embodiments, the preprocessing may further include segmenting the first positron emission tomography (PET) scan 210a and the first computed tomography (CT) scan 220a to generate the first tumor mask 230a as well as segmenting the second positron emission tomography (PET) scan 210b, and the second computed tomography (CT) scan 220b to generate the second tumor mask 230b. For example, in some cases, the preprocessing engine 111 may apply a segmentation model 112 to segment the first positron emission tomography (PET) scan 210a and the first computed tomography (CT) scan 220a by at least identifying a first plurality of pixels corresponding to one or more lesions present in the first positron emission tomography (PET) scan 210a and the first computed tomography (CT) scan 220a. Similarly, the preprocessing engine 111 may apply the segmentation model 112 to segment the second positron emission tomography (PET) scan 210b and the second computed tomography (CT) scan 220b by at least identifying a second plurality of pixels corresponding to one or more lesions present in the second positron emission tomography (PET) scan 210b and the second computed tomography (CT) scan 220b. As noted, each pixel in a positron emission tomography (PET) scan may be associated with an intensity value corresponding to a level of metabolic activity (e.g., standard update value (SUV)) while each corresponding pixel in a co-registered computed tomography (CT) scan may be associated with an intensity value corresponding to a tissue density or x-ray attenuation. Accordingly, in some cases, the aforementioned segmentation model may determine whether a pixel is a part of a lesion based on the level of metabolic activity and the tissue density (or x-ray attenuation) exhibited by the pixel and one or more neighboring pixels.

In some example embodiments, the segmentation model 112 may include one or more machine learning models trained to segment two-dimensional images and/or three-dimensional volumes. For example, in some cases, the segmentation model 112 may include one or more artificial neural networks such as convolutional neural networks, vision transformers, and/or the like. In instances where the segmentation model 112 includes one or more vision transformers, the segmentation model 112 may be applied to identify individual patches containing lesions before a threshold is applied to those patches to select pixels exhibiting a level of metabolic activity (e.g., standard uptake value (SUV)), a tissue density, and/or an X-ray attenuation satisfy one or more thresholds. For instance, a pixel may be identified as depicting a lesion if the standard uptake value (SUV) of the pixel exceeds a certain minimum value (e.g., 2.5 or 4), exceeds a certain minimum value relative to the standard uptake value (SUV) of the liver (e.g., 1.5 times the standard uptake value (SUV) of the liver plus 2 standard deviations from the standard uptake value (SUV) of the liver), a percentage of the maximum standard uptake value (SUVₘₐₓ) of the identified tumorous region, and/or the like.

In some cases, the segmentation model 112 may perform segmentation through object classification. In those instances, the segmentation model 112 may include one or more machine learning models (e.g., logistic regression models, tree-based classifiers, fully-connected neural networks, and/or the like) trained to perform object classification. To segment, for example, the first positron emission tomography (PET) scan 210a and the first computed tomography (CT) scan 220a, the preprocessing engine 111 may first apply thresholding to identify one or more objects present in the first positron emission tomography (PET) scan 210a and the first computed tomography (CT) scan 220a. For example, in some cases, the preprocessing engine 111 may identify, based at least on the intensity value of each pixel, pixels depicting objects in the first positron emission tomography (PET) scan 210a and the first computed tomography (CT) scan 220a. As noted, the intensity value of each pixel in a computed tomography (CT) scan corresponds to a tissue density or X-ray attenuation while the intensity value of a pixel in a positron emission tomography (PET) scan corresponds to a level of metabolic activity. Accordingly, in some cases, the preprocessing controller 110 may identify objects present in the first positron emission tomography (PET) scan 210a and the first computed tomography (CT) scan 220a by applying a first threshold on the intensity value of each pixel in the first positron emission tomography (PET) scan 210a and/or a second threshold on the intensity value of each pixel in the first computed tomography (CT) scan 210b. In doing so, the preprocessing engine 111 may identify objects exhibiting a threshold level of metabolic activity, a threshold level of tissue density, and/or a threshold level of X-ray attenuation. Upon identifying the objects that are present in the first positron emission tomography (PET) scan 210a and the first computed tomography (CT) scan 220a, the preprocessing engine 111 may apply the segmentation model 112 to classify each of the objects as either a lesion or not a lesion.

Referring again to FIG. 2, in some example embodiments, the analysis controller 110 may apply the longitudinal segmentation model 113 to update the first tumor mask 230a and the second tumor mask 230b. For example, in some cases, a first patch may be extracted from the first positron emission tomography (PET) scan 210a and the first computed tomography (CT) scan 220a to include the first lesion associated with the first tumor mask 230a while a second patch may be extracted from the second positron emission tomography (PET) scan 210b and the second computed tomography (CT) scan 220b to include the second lesion associated with the second tumor mask 230b. In some case, the longitudinal segmentation model 113 may ingest the first patch and the second patch instead of the first positron emission tomography (PET) scan 210a, the first computed tomography (CT) scan 220a, the second positron emission tomography (PET) scan 210b, and the second computed tomography (CT) scan 220b in their entirety in order to increase the signal-to-noise (SNR) associated with the pixels depicting the first lesion and the second lesion.

In some example embodiments, the longitudinal segmentation model 113 may update, based at least on the first patch and the second patch, the first tumor mask 230a and the second tumor mask 230b to generate the first updated tumor mask 240a and the second updated tumor mask 240b. For example, in some cases, the longitudinal segmentation model 113 may determine whether a pixel is a part of a lesion based on the level of metabolic activity and the tissue density (or x-ray attenuation) exhibited by the pixel and one or more neighboring pixels across the first timepoint and the second timepoint. In doing so, the longitudinal segmentation model 113 may update the first tumor mask 230a and/or the second tumor mask 230b including by updating the label assigned to one or more pixels in the first tumor mask 230a and/or the second tumor mask 230b. For instance, in some cases, a pixel previously classified (e.g., by the segmentation model) as being a part of a lesion is reclassified by the longitudinal segmentation model 113 as not being part of a lesion and a pixel previously classified (e.g., by the segmentation model) as not being a part of a lesion is reclassified by the longitudinal segmentation model 113 as being a part of a lesion.

Referring again to FIG. 2, the analysis controller 110 may include an assessment engine 115 that determines, based at least on the first updated tumor mask 240a and the second updated tumor mask 240b, a treatment response 180. For example, the treatment response 180 may be complete metabolic response (CMR) in instances where the lesions present in the first updated tumor mask 240a are absent from the second updated tumor mask 240b and no new lesions are present in the second updated tumor mask 240b. In instances where the some but not all of the lesions present in the first updated tumor mask 240a are present in the second updated tumor mask 240b and no new lesions are present in the second updated tumor mask 240b, the treatment response 180 may be partial metabolic response (PMR). In cases where the second updated tumor mask 240b includes one or more new lesions, the treatment response 180 may be progressive metabolic disease (PMD). Even without the presence of new lesions in the second updated tumor mask 240b, the treatment response 180 may still be progressive metabolic disease (PMD) in cases where the change in the level of metabolic activity exhibited by the lesions in the first updated tumor mask 240a and the same lesions in the second updated tumor mask 240b satisfy one or more thresholds.

Accordingly, in some cases, the assessment engine 115 may determine, based at least on the first updated tumor mask 240a and the second updated tumor mask 240b, the treatment response 180 as responder where, for example, the assessment engine 115 detects a complete metabolic response (CMR) or partial metabolic response. Alternatively, the assessment engine 115 may determine, based at last on the first updated tumor mask 240a and the second updated tumor mask 240b, the treatment response 180 as non-responder where, for example, the assessment engine 115 detects no metabolic response (NMR) or progressive metabolic disease (PMD). In some cases, the assessment engine 115 may determine, based at least on the first updated tumor mask 240a and the second updated tumor mask 240b, the treatment response 180 to be complete metabolic response (CMR), partial metabolic response (PMR), no metabolic response (NMR), or progressive metabolic disease (PMD). Alternatively, the assessment engine 115 may determine, based at least on the first updated tumor mask 240a and the second updated tumor mask 240b, the treatment response 180 to be complete metabolic response (CMR) or non-complete metabolic response (non-CMR). Furthermore, in some cases, in addition to or instead of the treatment response 180, the assessment engine 115 may determine, based at least on the first updated tumor mask 240a and the second updated tumor mask 240b, a progression of a disease, an overall survival (OS), and/or a progression free survival (PFS).

In some example embodiments, the assessment engine 115 may determine the treatment response 180 by at least determining, based at least on the first updated tumor mask 240a and the second updated tumor mask 240b, whether the second lesion associated with the second updated tumor mask 240b is a new lesion or a same lesion as the first lesion associated with the first updated tumor mask 240a. For example, the assessment engine 115 may determine, based at least on the first updated tumor mask 240a and the second updated tumor mask 240b, a distance between the first lesion and the second lesion. In some cases, the distance between the first lesion and the second lesion may be quantified by one or more of a maximum, a minimum, an average, a median, and/or a mode of the distances between a first plurality of pixels in the first updated tumor mask 240a and a second plurality of pixels in the second updated tumor mask 240b. In instances where the distance between the first lesion and the second lesion is determined to satisfy one or more thresholds (e.g., a minimum distance exceeding 10 millimeters), the assessment engine 115 may determine that the second lesion is a new lesion. Alternatively, where the distance between the first lesion and the second lesion fails to satisfy the one or more thresholds (e.g., a minimum distance that fails to exceed 10 millimeters), the assessment engine 115 may determine that the second lesion is a same lesion as the first lesion.

In some example embodiments, where the second lesion is identified as a new lesion, the assessment engine 115 may determine that the treatment response 180 is progressive metabolic disease (PMD). Alternatively, where the second lesion is identified as a same lesion as the first lesion, the assessment engine 115 may further determine the treatment response 180 based on a change in metabolic activity exhibited by the lesion between the first timepoint and the second timepoint. For example, in some cases, the assessment engine 115 may determine, based at least on the first updated tumor mask 240a and the first positron emission tomography (PET) scan 210a, a first level of metabolic activity exhibited by the lesion at the first timepoint. Moreover, the assessment engine 115 may determine, based at least on the second updated tumor mask 240b and the second positron emission tomography (PET) scan 210b, a second level of metabolic activity exhibited by the lesion at the second timepoint. In some cases, the level of metabolic activity exhibited by the lesion at each timepoint may correspond to a maximum, a minimum, a mean, a median, and/or a mode of the level of metabolic activity. For instance, the level of metabolic activity exhibited by the lesion at each timepoint may correspond to a maximum, a minimum, a median, a mean, and/or a mode value across the standard uptake values (SUV) associated with those pixels in a positron emission tomography (PET) scan from that timepoint identified by the corresponding updated tumor mask as being a part of the lesion.

In some cases, the change in metabolic activity exhibited by the lesion between the first timepoint and the second timepoint may correspond to a difference between the first level of metabolic activity exhibited by the lesion at the first timepoint and the second level of metabolic activity exhibited by the second timepoint. For example, the change in metabolic activity exhibited by the lesion between the first timepoint and the second timepoint may correspond to a difference in the maximum standard uptake value (SUVₘₐₓ) across the two timepoints. Alternatively and/or additionally, the level of metabolic activity exhibited by the lesion at each timepoint may correspond to a size of the lesion observed at each timepoint. Accordingly, in some cases, the change in metabolic activity exhibited by the lesion may also be determined based at least on a difference between a first size of the lesion at the first timepoint and a second size of the lesion at the second timepoint.

As noted, in some example embodiments, where the second lesion is identified as a same lesion as the first lesion, the assessment engine 115 may further determine the treatment response 180 based on a change in metabolic activity exhibited by the lesion between the first timepoint and the second timepoint. For example, in some cases, where the change in metabolic activity exhibited by the lesion between the first timepoint and the second timepoint satisfies a first threshold, the assessment engine 115 may determine that the treatment response 180 is progressive metabolic disease (PMD). Alternatively, in cases where the change in metabolic activity exhibited by the lesion between the first timepoint and the second timepoint satisfies a second threshold but not the first threshold, the assessment engine 115 may determine that the treatment response 180 is no metabolic response (NMR). Furthermore, in cases where the change in metabolic activity exhibited by the lesion between the first timepoint and the second timepoint satisfies neither the first threshold nor the second threshold, the assessment engine 115 may determine that the treatment response 180 is partial metabolic response (PMR).

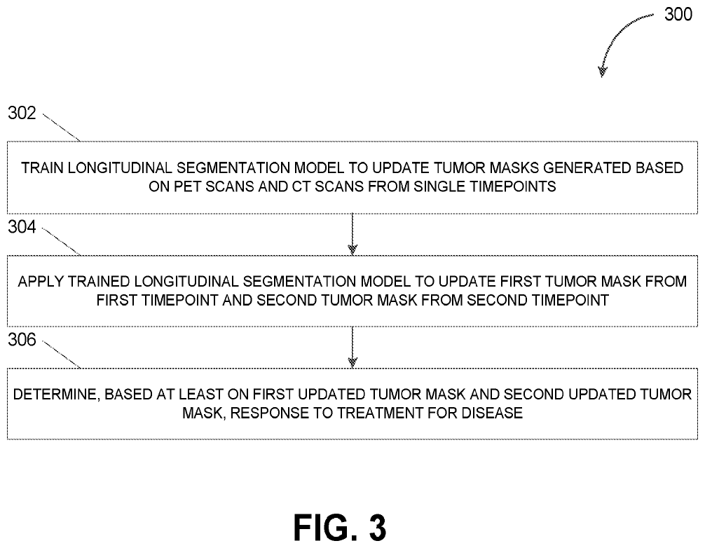

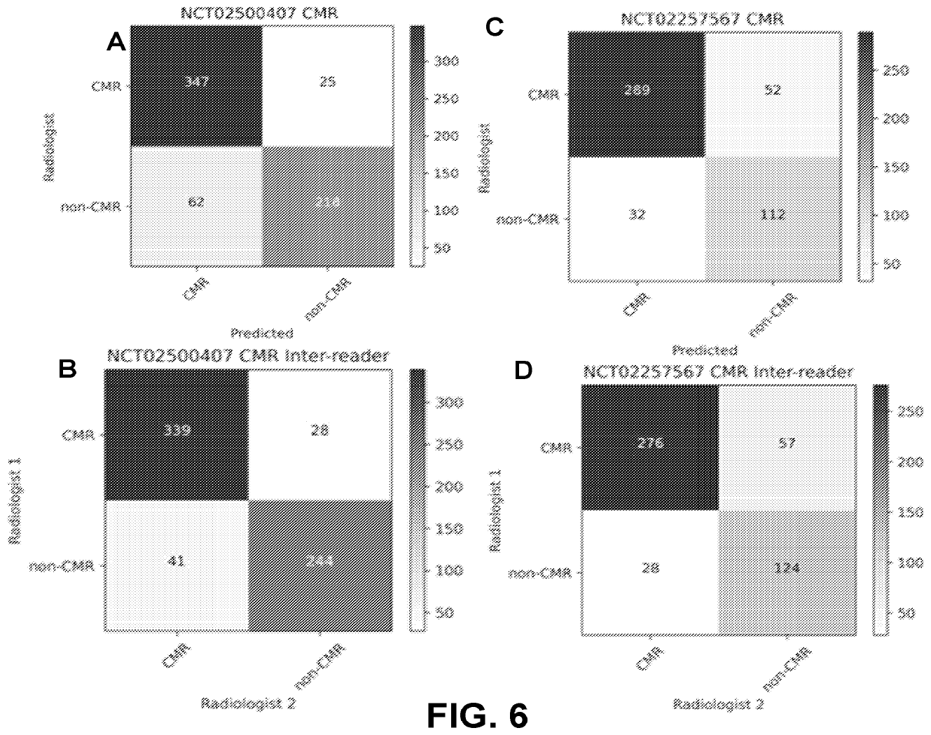

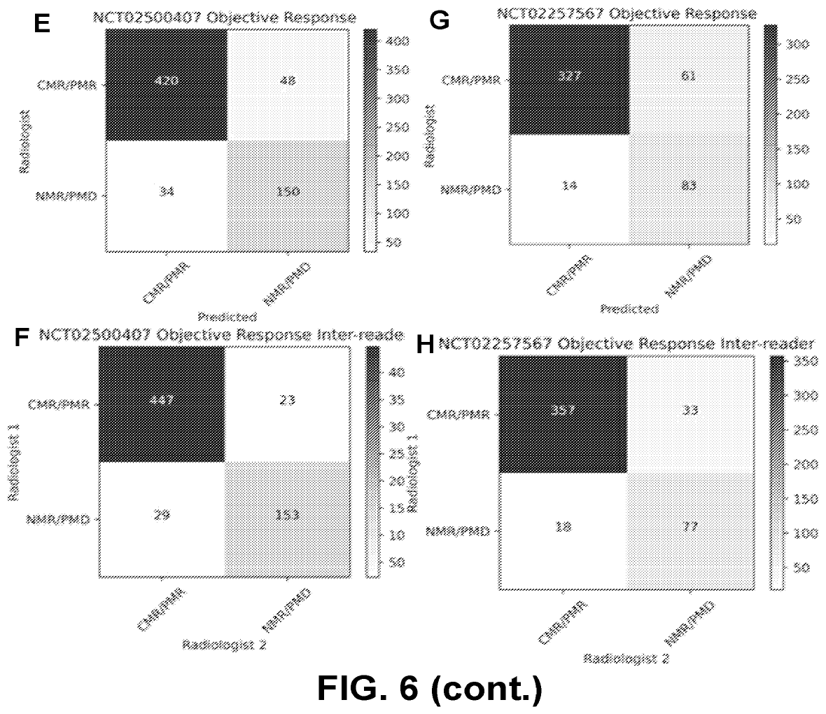

FIG. 3 depicts a flowchart illustrating an example of a process 300 for machine learning enabled longitudinal analysis of positron emission tomography (PET) and computed tomography (CT) scans, in accordance with some example embodiments. Referring to FIGS. 1-3, the process 300 may be performed by the analysis controller 110. The analysis controller 110 may perform the process 300 to determine, for example, the treatment response 180. As indicated by the performance metrics shown in FIGS. 6-7, 8A-G, 9, and 10, the analysis controller 110 performing the process 300 is able to achieve highly accurate results that are consistent with those determined by expert radiologists. The process 300 can be performed to achieve highly accurate results independently (e.g., without requiring expert radiologist intervention), meaning that process 300 provides a more efficient diagnostic solution that requires fewer resources than conventional techniques for analyzing positron emission tomography (PET) and computed tomography (CT) scans.

At 302, the analysis controller 110 may train the longitudinal segmentation model 113 to update tumor masks generated based on positron emission tomography (PET) scans and computed tomography (CT) scans from single timepoints. In some example embodiments, the analysis controller 110 may train, based at least on a training set, the longitudinal segmentation model 113 to update two or more tumor masks, each of which being generated from a positron emission tomography (PET) scan and a computed tomography (CT) scan from a single timepoint. In some cases, the training set may include one or more annotated training samples, each of which including a positron emission tomography (PET) scan and a computed tomography (CT) scan from two or more different timepoints as well as the corresponding ground truth tumor masks. Moreover, in some cases, each annotated training sample may include a region (e.g., a head and neck region, a chest region, an abdomen and pelvis region, and/or the like) extracted from the positron emission tomography (PET) scan and the computed tomography (CT) scan from two or more different timepoints and the corresponding ground truth tumor masks. In this context, each pixel in a ground truth tumor mask may be associated with a ground truth label having a first value (e.g., “1”) to indicate that the pixel is a part of a lesion or a second value (e.g., “0”) to indicate that the pixel Is not a part of a lesion.

At 304, the analysis controller 110 may apply the trained longitudinal segmentation model 113 to update a first tumor mask from a first timepoint and a second tumor mask from a second timepoint. In some example embodiments, as shown in FIG. 2, the analysis controller 110 may apply the trained longitudinal segmentation model 113 to update, based at least on the first positron emission tomography (PET) scan 210a and the first computed tomography (CT) scan 220a from the first timepoint and the second positron emission tomography (PET) scan 210b and the second computed tomography (CT) scan 220b from the second timepoint, the first tumor mask 230a from the first timepoint and the second tumor mask 230b from the second timepoint. As will be described in more detail below, the first tumor mask 230a may be generated based on the first positron emission tomography (PET) scan 210a and the first computed tomography (CT) scan 220a while the second tumor mask 230b may be generated based on the second positron emission tomography (PET) scan 210b and the second computed tomography (CT) scan 220b. The longitudinal segmentation model 113 may leverage information (e.g., level of metabolic activity, tissue density (or x-ray attenuation), and/or the like) from across multiple timepoints in order refine each of the first tumor mask 230a and the second tumor mask 230b to reduce false positives in which one or more pixels that are not part of a lesion are incorrectly identified as such in the first tumor mask 230a and/or the second tumor mask 230b.

At 306, the analysis controller 110 may determine, based at least on the first updated tumor mask and the second updated tumor mask, a response to a treatment for a disease. In some example embodiments, FIG. 2 shows that the assessment engine 115 may determine, based at least on the first updated tumor mask 240a and the second updated tumor mask 240b, the treatment response 180. As will be described in more detail below, the assessment engine 115 may determine the treatment response 180 based at least on whether the second lesion associated with the second updated tumor mask 240b is a new lesion or a same lesion as the first lesion associated with the first updated tumor mask 240a. In the event the assessment engine 115 determines that the second lesion associated with the second updated tumor mask 240b is not a new lesion but a same lesion as the first lesion associated with the first updated tumor mask 240a, the assessment engine 115 may determine the treatment response 180 based on a change in metabolic activity as determined based on the first updated tumor mask 240a, the first positron emission tomography (PET) scan 210a, the second updated tumor mask 240b, and the second positron emission tomography (PET) scan 210b.

In some cases, in addition to or instead of the treatment response 180, the assessment engine 115 may determine, based at least on the first updated tumor mask 240a and the second updated tumor mask 240b, a progression of a disease such as non-Hodgkin lymphoma (NHL) or another fluorodeoxyglucose avid (FDG-avid) cancer observable in positron emission tomography (PET) scans, In some cases, the assessment engine 115 may also determine, based at least on the first updated tumor mask 240a and the second updated tumor mask 240b, a change in tumor volume (e.g., total metabolic tumor volume (TMTV) and/or the like), which may in turn be indicative of the treatment response 180 and/or disease progression. Alternatively and/or additionally, in some cases, the assessment engine 115 may determine, based at least on the first updated tumor mask 240a and the second updated tumor mask 240b, a variance in a first change in metabolic activity and/or a second change in tumor volume between the first timepoint and the second timepoint across different lesions.

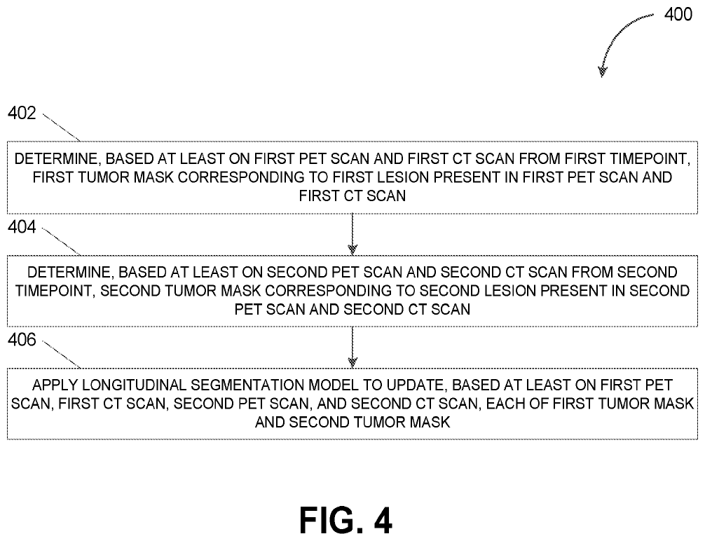

FIG. 4 depicts a flowchart illustrating another example of a process 400 for machine learning enabled longitudinal analysis of positron emission tomography (PET) and computed tomography (CT) scans, in accordance with some example embodiments. Referring to FIGS. 1-4, the process 400 may be performed by the analysis controller 110 and may implement, for example, operation 304 of the process 300.

At 402, the analysis controller 110 may determine, based at least on a first positron emission tomography (PET) scan and a first computed tomography (CT) scan from a first timepoint, a first tumor mask corresponding to a first lesion present in the first positron emission tomography (PET) scan and the first computed tomography (CT) scan. For instance, in the example shown in FIG. 2, the preprocessing engine 111 may determine, based at least on the first positron emission tomography (PET) scan 210a and the first computed tomography (CT) scan 220a, the first tumor mask 230a corresponding to the first lesion depicted in each of the first positron emission tomography (PET) scan 210a and the first computed tomography (CT) scan 220a. In some cases, the preprocessing engine 111 may determine the first tumor mask 230a by at least applying a segmentation model to the first positron emission tomography (PET) scan 210a and the first computed tomography (CT) scan 220a. Moreover, in some cases, the preprocessing controller 110 may align the first positron emission tomography (PET) scan 210a and the first computed tomography (CT) scan 220a to generate a first superimposed (or co-registered) image (e.g., a first PET-CT scan). Alternatively and/or additionally, the preprocessing controller 110 may extract, from the first positron emission tomography (PET) scan 210a aligned with the first computed tomography (CT) scan 220a, a first patch including the first lesion for ingestion by the longitudinal segmentation model 113.

At 404, the analysis controller 110 may determine, based at least on a second positron emission tomography (PET) scan and a second computed tomography (CT) scan from a second timepoint, a second tumor mask corresponding to a second lesion present in the second positron emission tomography (PET) scan and the second computed tomography (CT) scan. In the example shown in FIG. 2, the preprocessing engine 111 may determine, based at least on the second positron emission tomography (PET) scan 210b and the second computed tomography (CT) scan 220b, the second tumor mask 230b corresponding to the second lesion depicted in each of the second positron emission tomography (PET) scan 210b and the second computed tomography (CT) scan 220b. As with the first tumor mask 230a, in some cases, the preprocessing engine 111 may determine the second tumor mask 230b by at least applying a segmentation model to the second positron emission tomography (PET) scan 210b and the second computed tomography (CT) scan 220b. Furthermore, in some cases, the preprocessing controller 110 may also align the second positron emission tomography (PET) scan 210b and the second computed tomography (CT) scan 220b to generate a second superimposed (or co-registered) image (e.g., a second PET-CT scan). Alternatively and/or additionally, the preprocessing controller 110 may extract, from the second positron emission tomography (PET) scan 210b aligned with the second computed tomography (CT) scan 220b, a second patch including the second lesion for ingestion by the longitudinal segmentation model 113.

At 406, the analysis controller 110 may apply the longitudinal segmentation model 113 to update, based at least on the first positron emission tomography (PET) scan, the first computed tomography (CT) scan, the second positron emission tomography (PET) scan, and the second computed tomography (CT) scan, each of the first tumor mask and the second tumor mask. For instance, in the example shown in FIG. 2, the analysis controller 110 may apply the longitudinal segmentation model 113 to update, based at least on the first patch extracted from the first positron emission tomography (PET) scan 210a and the first computed tomography (CT) scan 220a and the second patch extracted from the second positron emission tomography (PET) scan 210b and the second computed tomography (CT) scan 220b, each of the first tumor mask 230a and the second tumor mask 230b. As noted, in some cases, the longitudinal segmentation model 113 may determine whether a pixel is a part of a lesion based on the level of metabolic activity and the tissue density (or x-ray attenuation) exhibited by the pixel and one or more neighboring pixels across the first timepoint and the second timepoint. Accordingly, the longitudinal segmentation model 113 may update the first tumor mask 230a and/or the second tumor mask 230b including by updating the label assigned to one or more pixels in the first tumor mask 230a and/or the second tumor mask 230b. For example, in some cases, this updating may include the longitudinal segmentation model 113 reclassifying a pixel previously classified (e.g., by the segmentation model) as being a part of a lesion as not being part of a lesion. Alternatively and/or additionally, this updating may include the longitudinal segmentation model 113 reclassifying a pixel previously classified (e.g., by the segmentation model) as not being a part of a lesion as being a part of a lesion.

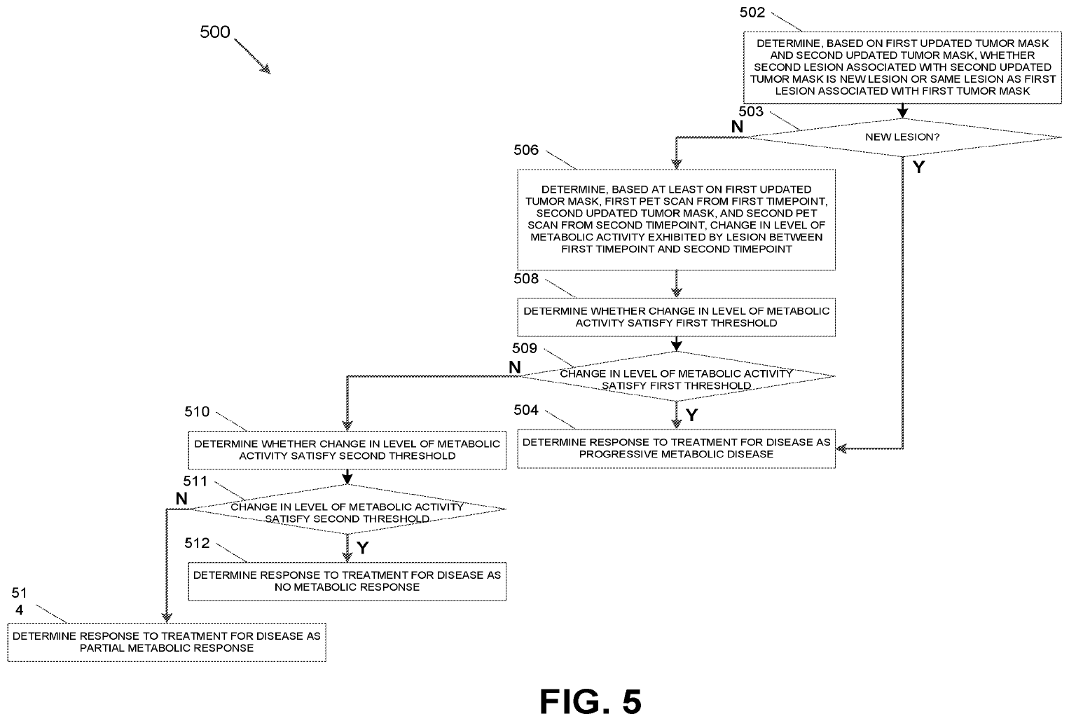

FIG. 5 depicts a flowchart illustrating another example of a process 500 for machine learning enabled longitudinal analysis of positron emission tomography (PET) and computed tomography (CT) scans, in accordance with some example embodiments. Referring to FIGS. 1-3 and 5, the process 500 may be performed by the analysis controller 110 and may implement, for example, operation 306 of the process 300.

In some cases, the process 500 may implement a Lugano classification process. Accordingly, in some cases, the treatment response 180 that is generated by the analysis controller 110 may conform to the Lugano classification (or tumor staging) paradigm, which includes identifying a patient's positron emission tomography (PET) and computed tomography (CT) scans as depicting progressive metabolic disease (PMD) (or non-complete metabolic response (non-CMR)), no metabolic response (NMR), or partial metabolic response (PMR). The performance metrics shown in FIGS. 6-7, 8A-G, 9, and 10 indicate that the analysis controller 110 performing the process 500 is able to achieve highly accurate results. That is, the Lugano classification resulting from the analysis controller 110 performing the process 500 are consistent with the determinations made by expert radiologists. Furthermore, the process 500 can be performed to achieve highly accurate results independently (e.g., without requiring expert radiologist intervention) and is therefore more expedient, efficient, and requires fewer resources than conventional techniques for analyzing positron emission tomography (PET) and computed tomography (CT) scans. The speed and efficiency of the process 500 may expedite many downstream clinical tasks including treatment decisions. In some cases, the treatment response 180 generated by the process 500 may be applied towards generating immediate treatment decisions, thereby eliminating a critical bottleneck in conventional clinical workflows.

As described in more details below, the process 500, which leverages insights derived from machine learning based analysis of longitudinal positron emission tomography (PET) and computed tomography (CT) scans, can be performed to generate the more granular Lugano classifications than conventional techniques, particularly those that merely examines data from a single timepoint. For example, the process 500 may be performed to provide an accurate and precise differentiation between progressive metabolic disease (PMD) (or non-complete metabolic response (non-CMR)), no metabolic response (NMR), and partial metabolic response (PMR), which may be more insightful than a binary classification (e.g., responder and non-responder). The accuracy and precision of the treatment response 180 generated by the process 500 means that the process 500 also improves the accuracy and precision of downstream clinical tasks, such as the identification of relapse and refractory patients, and treatment decisions, which rely on the outputs of the process 500.

At 502, the analysis controller 110 may determine, based at least on a first updated tumor mask from a first timepoint and a second updated tumor mask from a second timepoint, whether a second lesion associated with the second updated tumor mask is a new lesion or a same lesion as a first lesion associated with the first updated tumor mask. In some example embodiments, the assessment engine 115 may determine, based at least on the first updated tumor mask 240a from the first timepoint and the second updated tumor mask 240b from the second timepoint, a distance between the first lesion associated with the first updated tumor mask 240a and the second lesion associated with the second updated tumor mask 240b. In some cases, the distance between the first lesion and the second lesion may be quantified by one or more of a maximum, a minimum, an average, a median, and/or a mode of the distances between a first plurality of pixels in the first updated tumor mask 240a and a second plurality of pixels in the second updated tumor mask 240b. Whether the second lesion associated with the second updated tumor mask 240b is a new lesion or a same lesion as the first lesion associated with the first updated tumor mask 240a may be determined based on whether the distance between the first lesion and the second lesion satisfy one or more thresholds. For example, in some cases, the assessment engine 115 may determine that the second lesion is a new lesion if the distance (e.g., the maximum distance and/or the like) between the first lesion and the second lesion exceed a threshold value (e.g., 10 millimeters and/or the like). Alternatively, the assessment engine 115 may determine that the second lesion is not a new lesion but a same lesion as the first lesion associated with the first updated tumor mask 240a if the distance (e.g., the maximum distance and/or the like) between the first lesion and the second lesion does not exceed the threshold value (e.g., 10 millimeters and/or the like).

At 503-Y, the analysis controller 110 may identify the second lesion associated with the second updated tumor mask as a new lesion. As noted, in some cases, the assessment engine 115 may determine that the second lesion associated with the second updated tumor mask 240b is a new lesion and not a same lesion as the first lesion associated with the first updated tumor mask 240a if the distance between the first lesion and the second lesion satisfy one or more thresholds (e.g., a maximum distance between the first lesion and second lesion exceeds 10 millimeters and/or the like). Accordingly, at 504, the analysis controller 110 may determine the response to a treatment for a disease as progressive metabolic disease (PMD). For example, in instances where the second lesion associated with the second updated tumor mask 240b is identified as a new lesion, the assessment engine 115 may determine that the treatment response 180 is progressive metabolic disease (PMD) or, in some cases, non-complete metabolic response (non-CMR).

Alternatively, at 503-N, the analysis controller 110 may identify the second lesion associated with the second updated tumor mask not as a new lesion but as a same lesion as the first lesion associated with the first updated tumor mask. For example, in some cases, the assessment engine 115 may determine that the second lesion associated with the second updated tumor mask 240b is not a new lesion but a same lesion as the first lesion associated with the first updated tumor mask 240a if the distance between the first lesion and the second lesion fails to satisfy one or more thresholds (e.g., a maximum distance between the first lesion and second lesion does not exceed 10 millimeters and/or the like). As such, at 506, the analysis controller 110 may determine, based at least on the first updated tumor mask, a first positron emission tomography (PET) scan from the first timepoint, the second updated tumor mask, and a second positron emission tomography (PET) scan from the second timepoint, a change in a level of metabolic activity exhibited by the lesion between the first timepoint and the second timepoint. For instance, in some cases, the assessment engine 115 may determine the change in the level of metabolic activity exhibited by the lesion between the first timepoint and the second timepoint by at least determining, based at least on the first updated tumor mask 240a and the first positron emission tomography (PET) scan 210a, a first level of metabolic activity exhibited by the lesion at the first timepoint. Moreover, in some cases, the assessment engine 115 may determine the change in the level of metabolic activity exhibited by the lesion between the first timepoint and the second timepoint by at least determining, based at least on the second updated tumor mask 240b and the second positron emission tomography (PET) scan 210b, a second level of metabolic activity exhibited by the lesion at the second timepoint. The change in the level of metabolic activity exhibited by the lesion between the first timepoint and the second timepoint may correspond to a difference between the first level of metabolic activity and the second level of metabolic activity. It should be appreciated that in some instances, the level of metabolic activity exhibited by the lesion at any one timepoint may be quantified by a maximum, a minimum, an average, a median, and/or a mode of the level of metabolic activity (e.g., standardized uptake value (SUV)) exhibited by each pixel in the positron emission tomography (PET) scan identified by the corresponding updated tumor mask as being a part of the lesion. In some cases, the change in the level of metabolic activity ΔSUV exhibited by the lesion between the first timepoint and the second timepoint may be determined based on Equation (1) below

$$Δ SUV = \frac{SUV {max}_{t 2} - SUV {max}_{t 1}}{SUV {max}_{t 2}} \qquad ( 1 )$$

wherein SUVmaxₜ₁ denotes the first level of metabolic activity at the first timepoint t1 (e.g., screening, prior to treatment, and/or the like) and SUVmaxₜ₁ denotes the second level of metabolic activity at the second timepoint t2 (e.g., follow-up, subsequent to treatment, and/or the like).

At 508, the analysis controller 110 may determine whether the change in the level of metabolic activity exhibited by the lesion between the first timepoint and the second timepoint satisfies a first threshold. For example, in some cases, the assessment engine 115 may determine whether the change in the level of metabolic activity exhibited by the lesion between the first timepoint and the second timepoint, such as a difference between a first maximal standard uptake value (SUVmaxₜ₁) from the first timepoint and a second maximal standard uptake value (SUVmaxₜ₂) from the second timepoint, satisfies a first threshold value (e.g., ΔSUV>0.5).

At 509-Y, the analysis controller 110 may determine that the change in the metabolic activity exhibited by the lesion between the first timepoint and the second timepoint satisfies the first threshold. For example, in some cases, the assessment engine 115 may determine that the change in the level of metabolic activity exhibited by the lesion between the first timepoint and the second timepoint, such as the difference between a first maximal standard uptake value (SUVmaxₜ₁) from the first timepoint and a second maximal standard uptake value (SUVmaxₜ₂) from the second timepoint, satisfies the first threshold value (e.g., ΔSUV>0.5). Accordingly, the process 500 may resume at operation 504 where the analysis controller 110 determines the response to the treatment for the disease as progressive metabolic disease (PMD). For example, in instances where the change in the level of metabolic activity between the first timepoint and the second timepoint is determined to satisfy the first threshold value (e.g., ΔSUV>0.5), the assessment engine 115 may determine that the treatment response 180 is progressive metabolic disease (PMD) or, in some cases, non-complete metabolic response (non-CMR).

Alternatively, at 509-N, the analysis controller 110 may determine that the change in the metabolic activity exhibited by the lesion between the first timepoint and the second timepoint fails to satisfy the first threshold. For example, in some cases, the assessment engine 115 may determine that the change in the level of metabolic activity between the first timepoint and the second timepoint fails to satisfy the first threshold value (e.g., ΔSUV>0.5). Accordingly, at 510, the analysis controller 110 may determine whether the change in the metabolic activity exhibited by the lesion between the first timepoint and the second timepoint satisfies a second threshold. For instance, in instances where the change in the level of metabolic activity between the first timepoint and the second timepoint fails to satisfy the first threshold value (e.g., ΔSUV>0.5), the assessment engine 115 may further determine whether the change in the level of metabolic activity between the first timepoint and the second timepoint satisfy a second threshold (e.g., ΔSUV>−0.25).

At 511-Y, the analysis controller 110 may determine that the change in the metabolic activity exhibited by the lesion between the first timepoint and the second timepoint satisfies the second threshold. For example, in some cases, the assessment engine 115 may determine that the change in the level of metabolic activity exhibited by the lesion between the first timepoint and the second timepoint, such as the difference between the first maximal standard uptake value (SUVmaxₜ₁) from the first timepoint and the second maximal standard uptake value (SUVmaxₜ₂) from the second timepoint, satisfies the second threshold value but not the first threshold value (e.g., 0.5>ΔSUV>−0.25). As such, at 512, the analysis controller 110 may determine the response to the treatment for the disease as no metabolic response (NMR). For instance, in cases where the change in the level of metabolic activity exhibited by the lesion between the first timepoint and the second timepoint satisfies the second threshold value but not the first threshold value (e.g., 0.5>ΔSUV>−0.25), the assessment engine 115 may determine that the treatment response 180 is no metabolic response (NMR).

Alternatively, at 511-N, the analysis controller 110 may determine that the change in the metabolic activity exhibited by the lesion between the first timepoint and the second timepoint fails to satisfy the second threshold. For example, in some cases, the assessment engine 115 may determine that the change in the level of metabolic activity exhibited by the lesion between the first timepoint and the second timepoint, such as the difference between the first maximal standard uptake value (SUVmaxₜ₁) from the first timepoint and the second maximal standard uptake value (SUVmaxₜ₂) from the second timepoint, fails to satisfy the second threshold value in addition to the first threshold value (e.g., ΔSUV<−0.25). Accordingly, at 514, the analysis controller 110 may determine the response to the treatment for the disease as partial metabolic response (PMR). For instance, in cases where the assessment engine 115 determines that the change in the level of metabolic activity exhibited by the lesion between the first timepoint and the second timepoint satisfies neither the first threshold value nor the second threshold value (e.g., ΔSUV<−0.25), the assessment engine 115 may determine that the treatment response 180 is partial metabolic response (PMR).

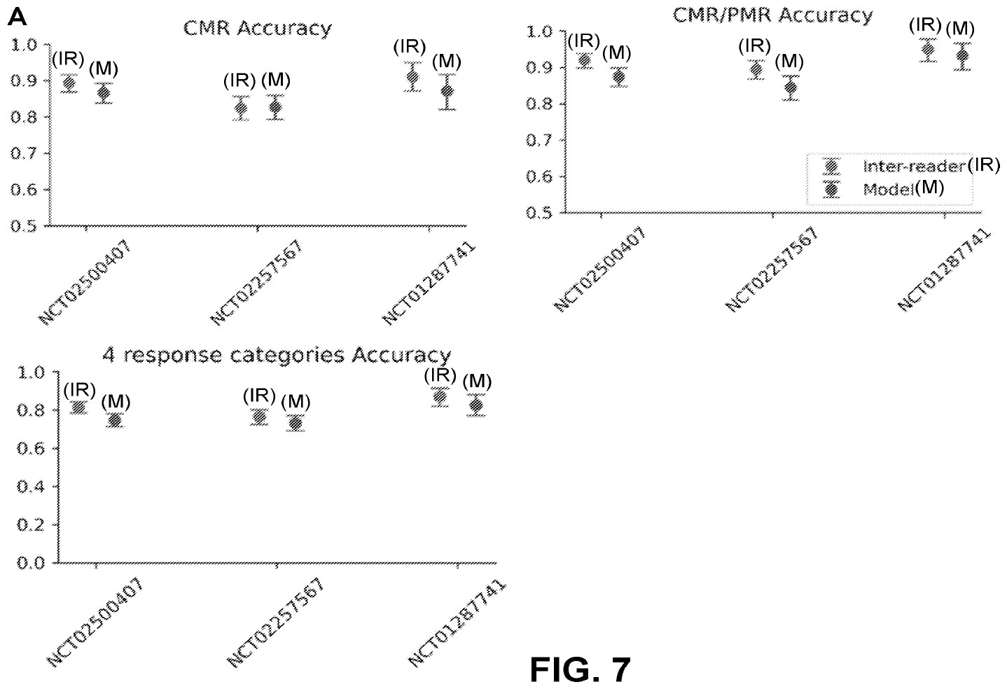

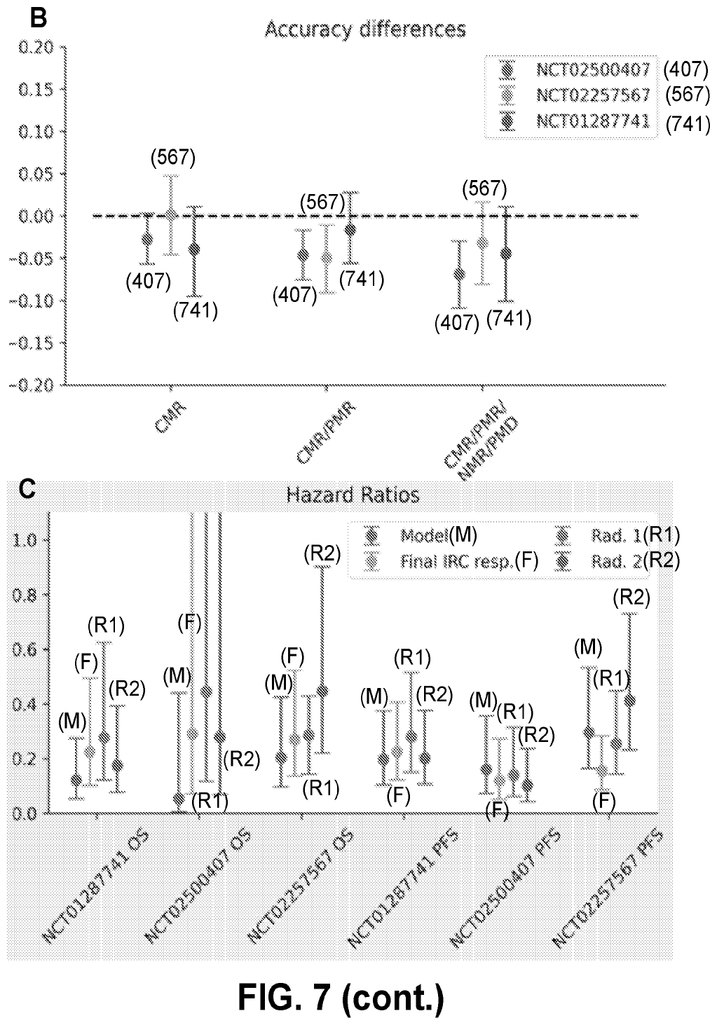

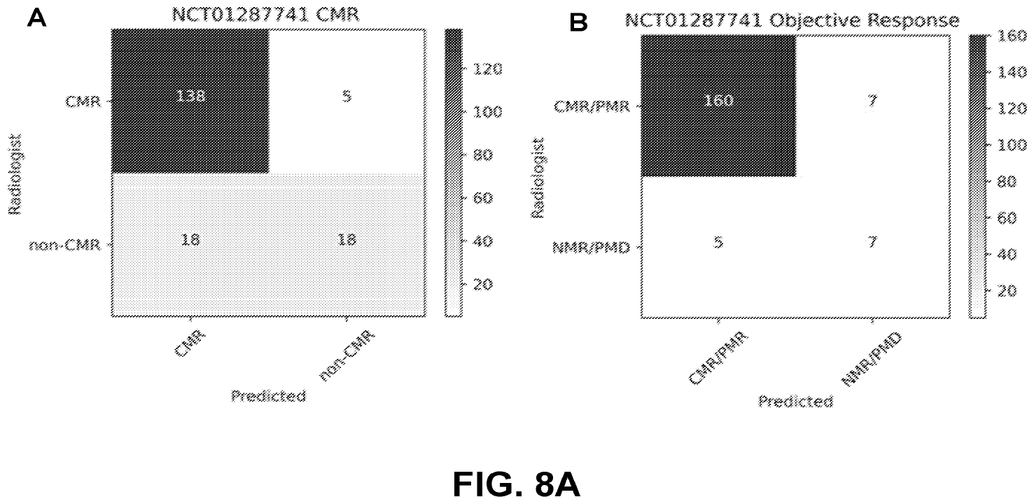

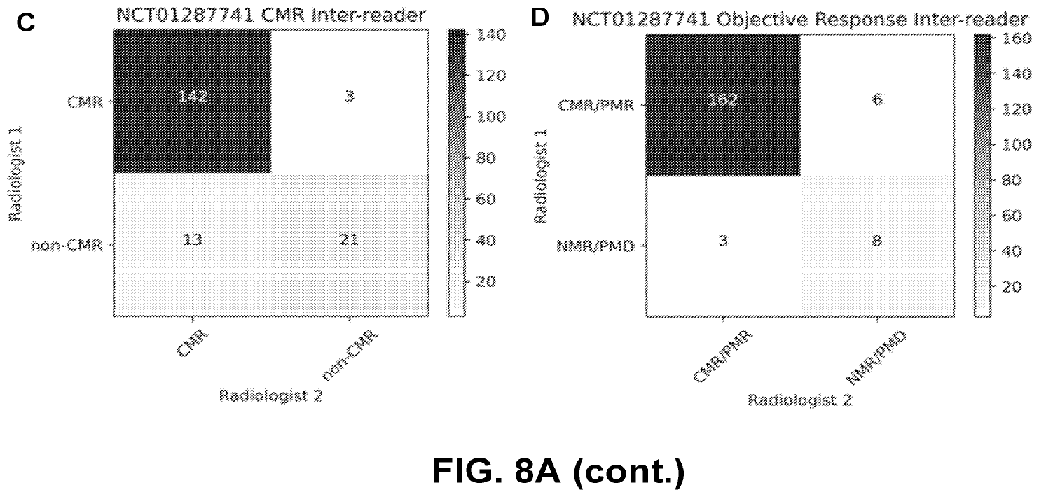

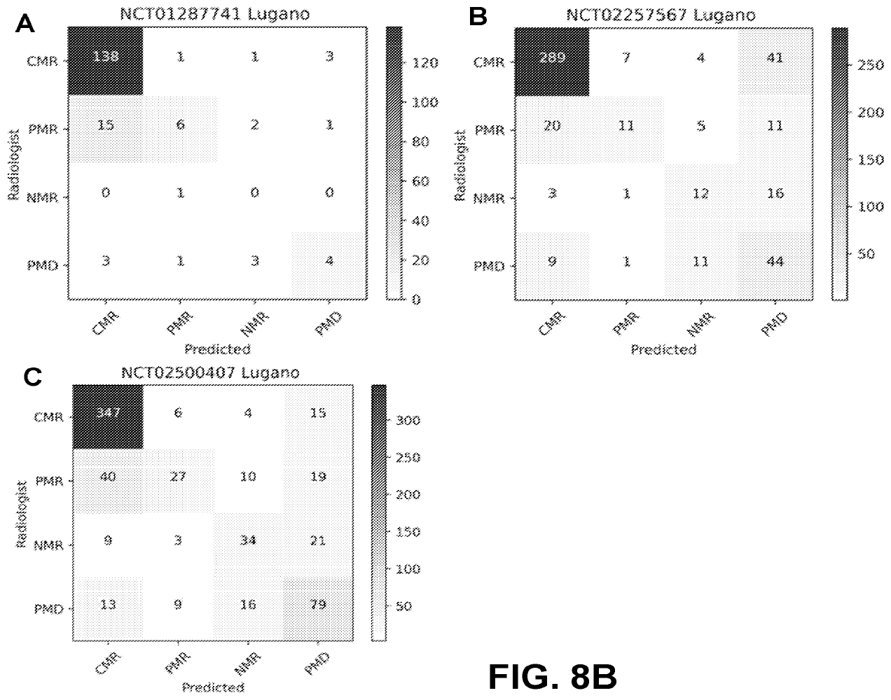

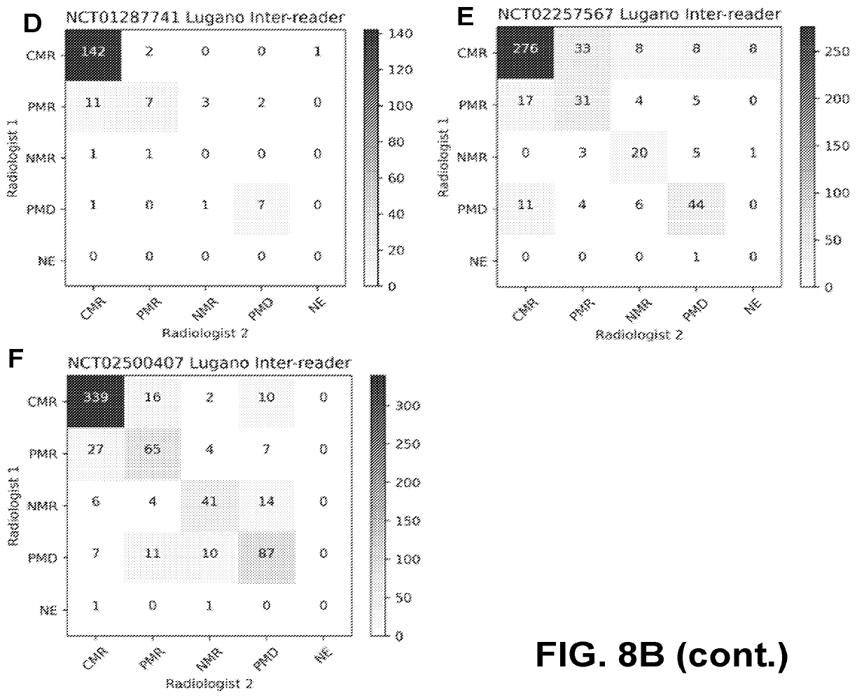

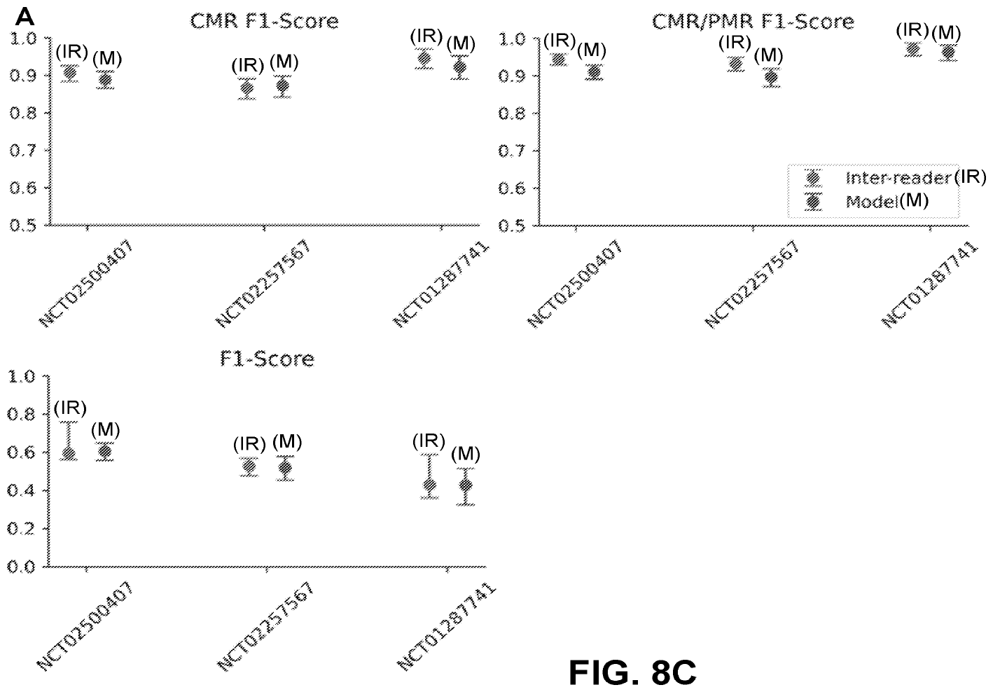

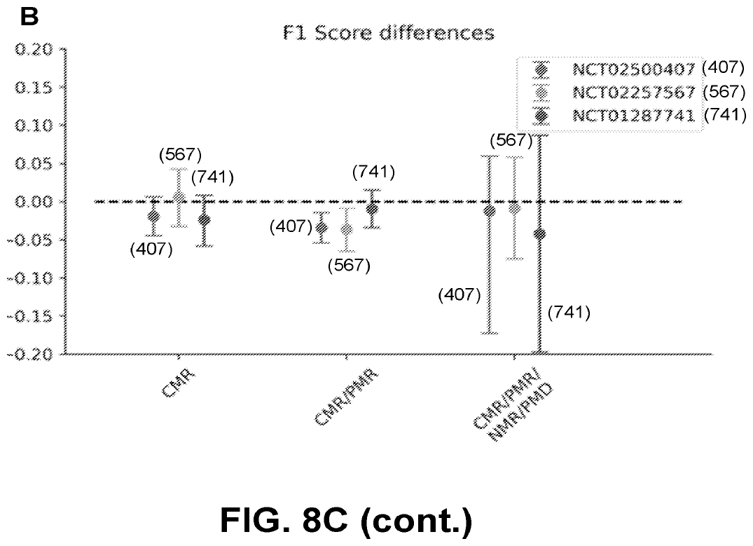

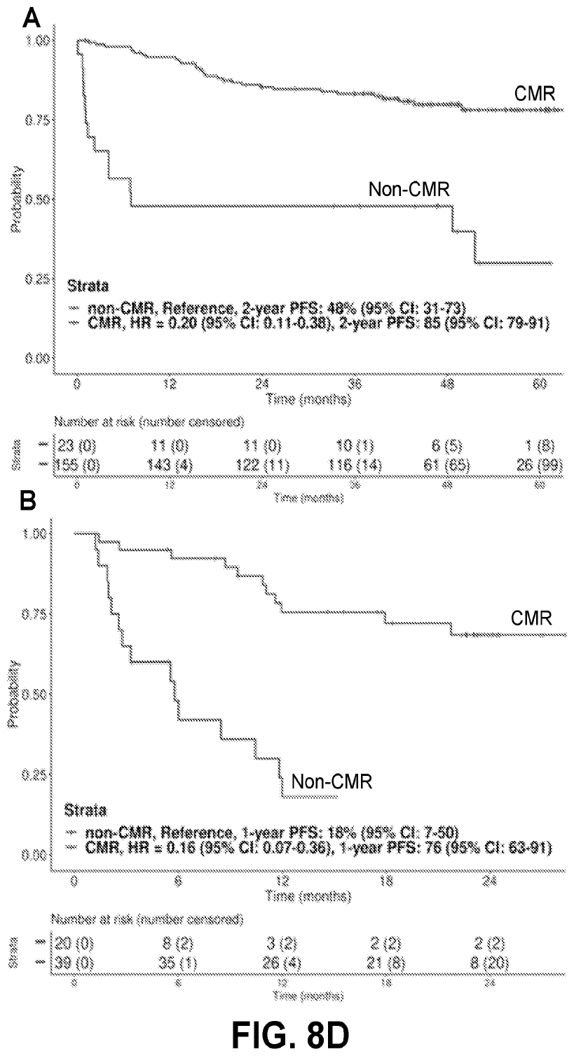

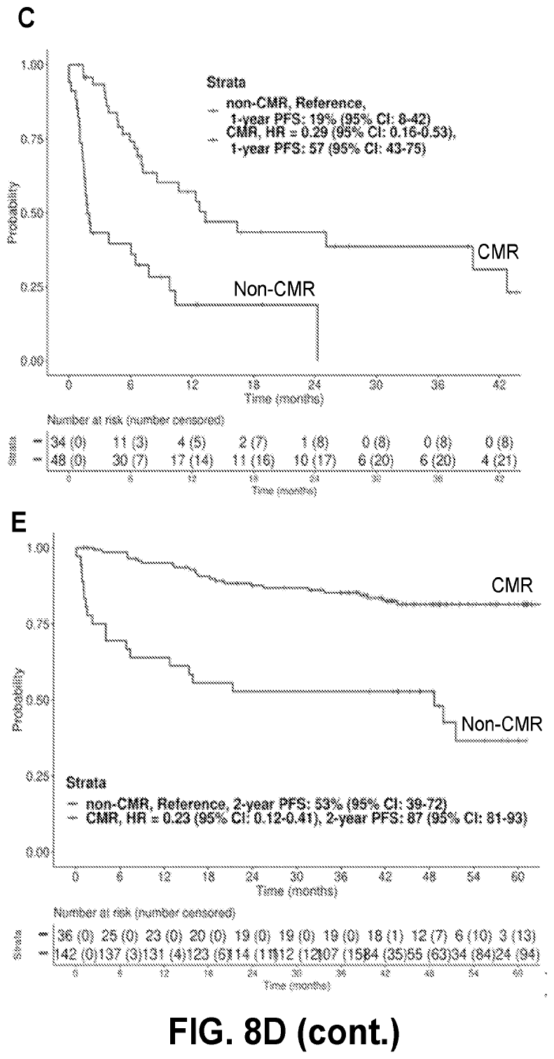

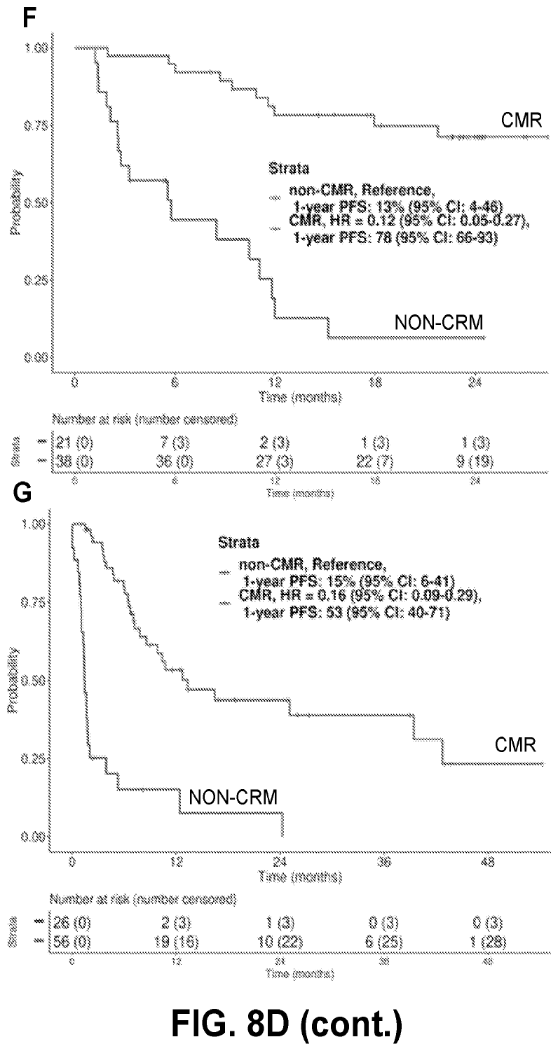

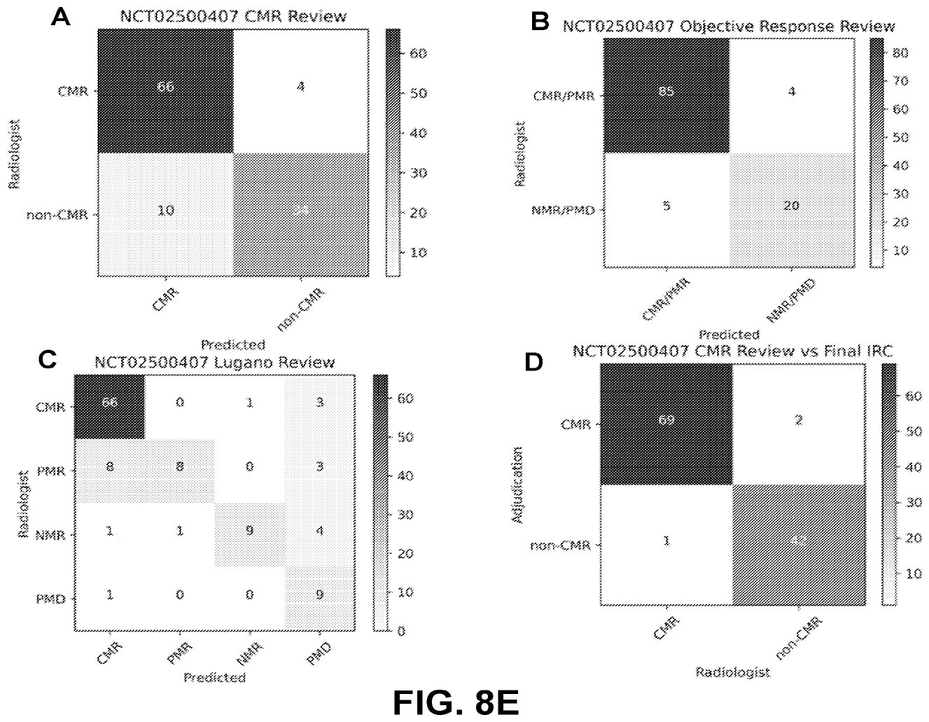

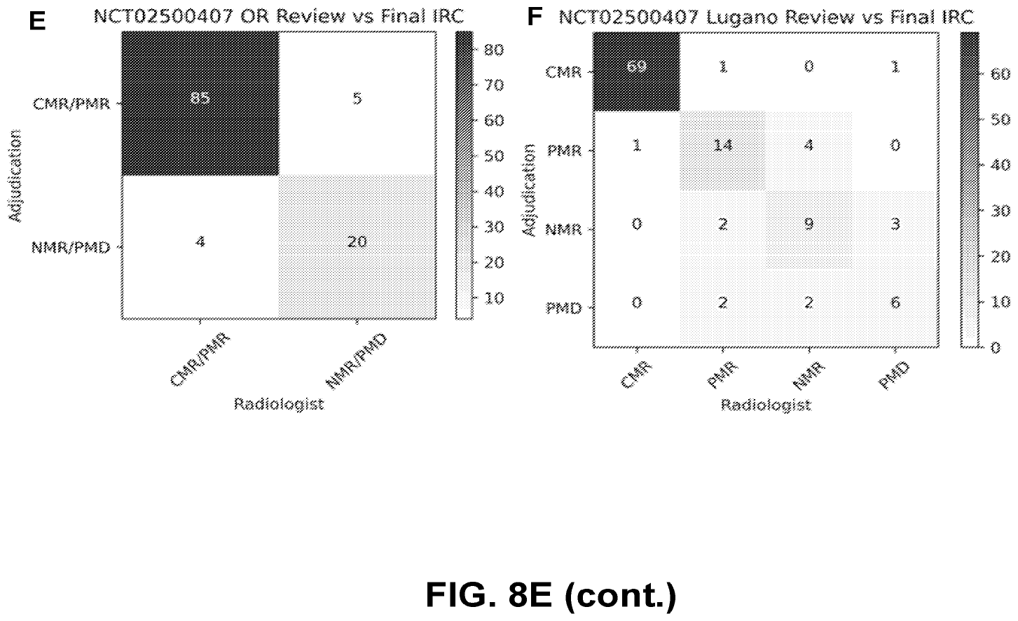

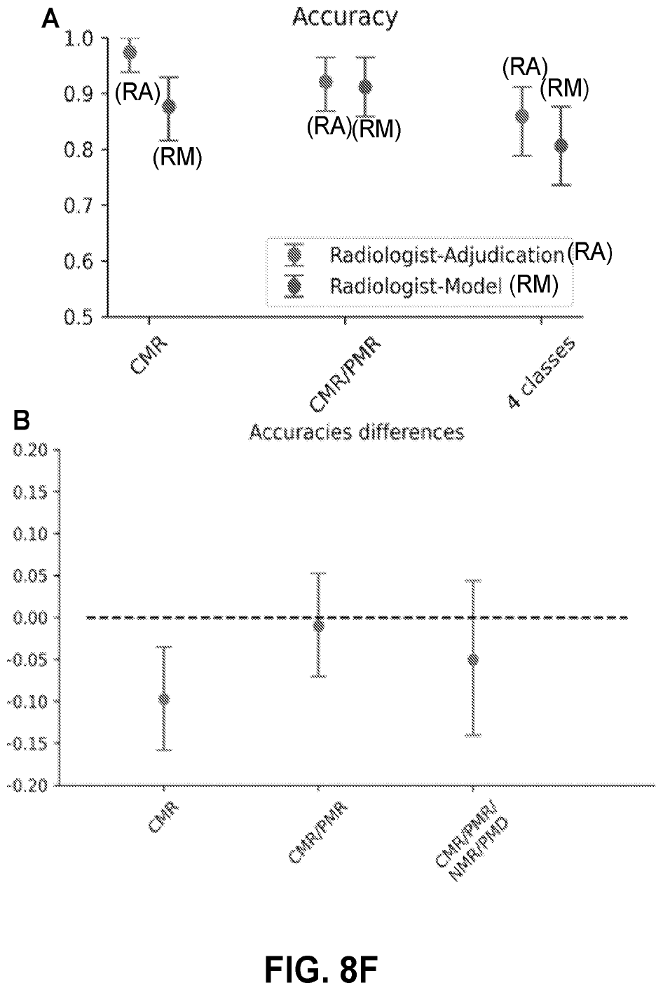

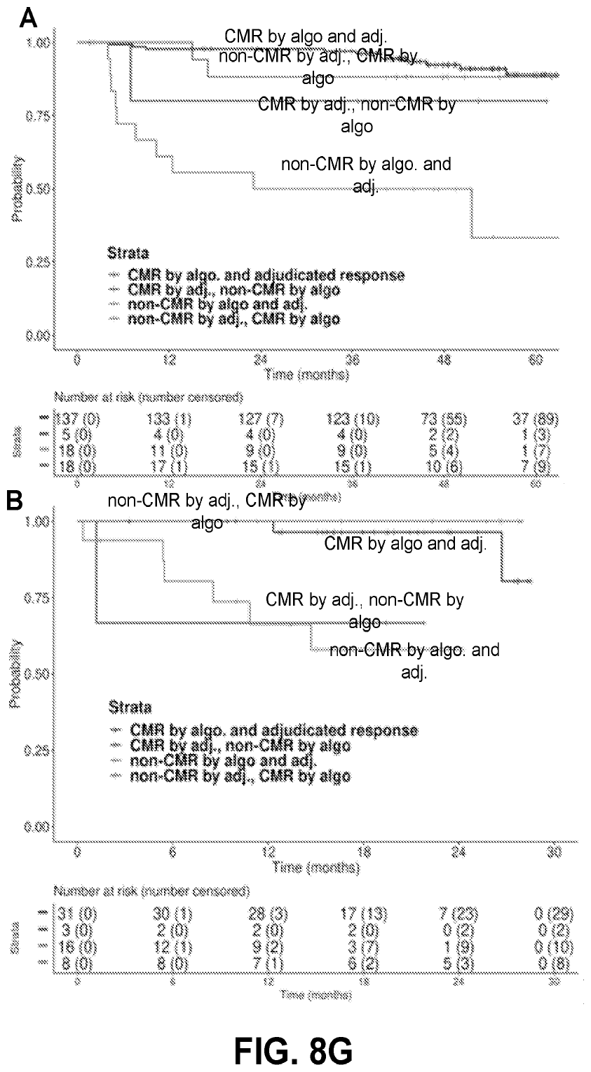

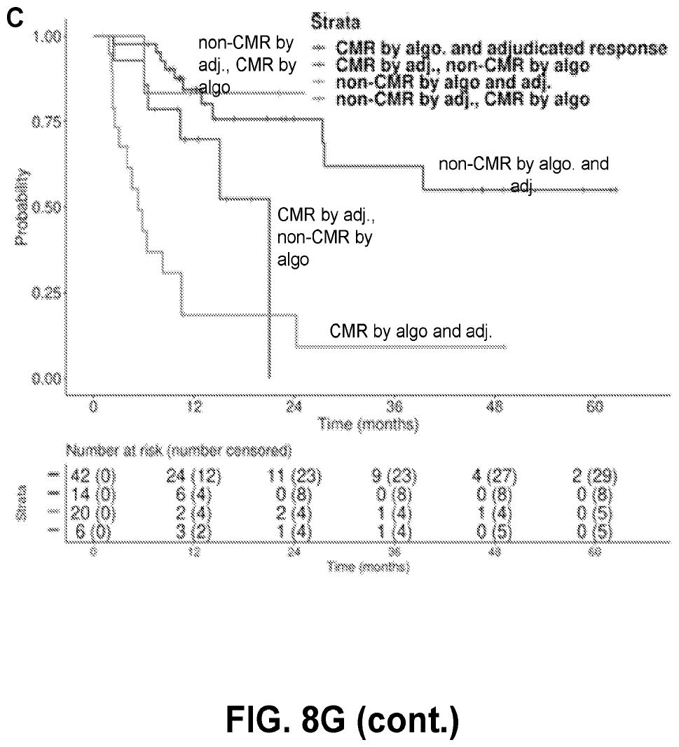

As noted, the analysis controller 110 may determine the treatment response 180 with better efficiency and a high level of accuracy, as measured by its consistency with the results generated by expert radiologists. The performance of the analysis controller 110 may be evaluated based on datasets from different populations and treatment protocols. The accuracy of the analysis controller 110 was assessed on 2,266 evaluable follow-up visits from 678 unique patients. The treatment response 180 determined by the analysis controller 110 shows a strong agreement with expert radiologist assessment of the same datasets. No statistically significant differences were observed between the performance of the analysis controller 110 compared to that of the final response from the expert radiologist committee versus the inter-radiologist agreement in six of the nine experiments (Table 1, FIG. 7(B)). The difference was less than 5% in two other experiments. The same comparisons with the F1-scores showed no significant differences in seven out of the nine comparisons and differences of less than 5% for the two other comparisons (FIG. 8C). Table 1 below shows the accuracy of the analysis controller 110 with final responses from expert radiologists on the test sets compared with the inter-radiologist agreement.

Table 1 includes three datasets: (1) GOYA (NCT01287741), (2) GO29365 (NCT02257567) and (3) GO29781 (NCT02500407). For GOYA, the first PET scan (baseline scan) was taken 1-35 days prior to treatment while the second PET scan (end of treatment scan) was taken 6-8 weeks after the last dose. For early discontinuation, the second PET scan was taken 4-8 weeks after the last dose. For GO29365, the first PET scan (baseline scan) was taken prior to treatment and second PET scans (interim scans) were taken 6 weeks during treatment, 3 months during treatment and every 3 months after while treatment was ongoing. Additionally, second PET scan (follow-up scans) were taken every 3 months for 18 months and then every 12 months. For GO29781, a first scan (baseline scan) was taken prior to treatment. A second PET scan was taken at week 6 during treatment (interim scan), when treatment was complete (end of treatment scan), and every 6 months for 2 years (follow up scan).

TABLE 1

| | | | |
|---|---|---|---|
| N scans | CMR vs | Objective | CMR/PMR/ |
| (N patients) | non-CMR | Response | NMR/PMD |

GOYA Holdout (NCT01287741) (R-CHOP or G-CHOP)

| | | | | |
|---|---|---|---|---|
| Radiologists | 179 | 0.91 | 0.95 | 0.87 |
| | (179) | (0.87, 0.94) | (0.92, 0.98) | (0.82, 0.92) |
| Model | | 0.87 | 0.93 | 0.83 |
| | | (0.82, 0.92) | (0.90, 0.97) | (0.78, 0.88) |
| Difference | | −0.03 | −0.02 | −0.04 |
| | | (−0.09, 0.02) | (−0.06, 0.02) | (−0.11, 0.01) |

GO29781/NCT02500407 (mosunetuzumab +/− atezolizumab)

| | | | | |
|---|---|---|---|---|
| Radiologists | 652 | 0.89 | 0.92 | 0.82 |
| | (250) | (0.86, 0.92) | (0.90, 0.94) | (0.79, 0.84) |
| Model | | 0.87 | 0.87 | 0.75 |
| | | (0.84, 0.89) | (0.85, 0.90) | (0.71, 0.78) |
| Difference | | −0.02 | −0.05 | −0.07 |
| | | (−0.06, 0.00) | (−0.07, −0.02) | (−0.11, −0.03) |

GO29365/NCT02257567 (bendamustine + rituximab +/− polatuzumab vedotin)

| | | | | |
|---|---|---|---|---|
| Radiologists | 485 | 0.82 | 0.89 | 0.76 |
| | (249) | (0.78, 0.85) | (0.87, 0.92) | (0.73, 0.80) |
| Model | | 0.83 | 0.85 | 0.73 |
| | | (0.79, 0.86) | (0.81, 0.88) | (0.69, 0.77) |
| Difference | | 0.01 | −0.04 | −0.03 |
| | | (−0.05, 0.05) | (−0.09, −0.01) | (−0.08, 0.03) |

FIG. 6 depicts the accuracies for complete metabolic response (CMR) assessment made by the analysis controller 110 in (A) clinical trial dataset GO29781/NCT02500407 and (C) clinical trial dataset GO29365/NCT02257567 and objective response (OR) assessment by the analysis controller 110 in (E) clinical trial dataset GO29781/NCT02500407 and (G) in clinical trial dataset GO29365/NCT02257567 as compared with, respectively, inter-reader agreement for complete metabolic response (CMR) assessment in (B) clinical trial dataset GO29781/NCT02500407 and (D) clinical trial dataset GO29365/NCT02257567 and inter-reader agreement for objective response (OR) assessment in (F) clinical trial dataset GO29781/NCT02500407 and (H) clinical trial dataset GO29365/NCT02257567.

FIG. 7(A) depicts a comparison of the accuracies for the analysis controller 110 and the expert radiologists while FIG. 7(B) depicts error bars for the differences between the inter-reader concordance and the accuracy of the analysis controller 110 compared to the final response. FIG. 7(C) shows the overall survival (OS) and progression free survival (PFS) hazard ratios for patients within each dataset identified by the analysis controller 110 and the expert radiologists as exhibiting complete metabolic response (CMR) at end of treatment (e.g., non-complete metabolic response).

The agreement between the treatment response 180 determined by the analysis controller 110 and that of expert radiologists for complete metabolic response (CMR) prediction on clinical trial dataset GO29781/NCT02500407, clinical trial dataset GO29365/NCT02257567, and the GOYA holdout set (NCT01287741) (Table 1, FIGS. 6, 7(A)-(B), and 8A) were respectively 0.87 (95% Confidence Interval (CI): 0.84, 0.89), 0.83 (95% CI: 0.79, 0.86) and 0.87 (95% CI: 0.82, 0.92). These comparisons showed no statistically significant differences when compared to the inter-reader agreement, respectively estimated as −0.02 (95% CI: −0.06, 0.00); 0.01 (95% CI: −0.05, 0.05); and −0.03 (95% CI: −0.09, 0.02). The objective response (OR) and the four-response category accuracy and inter-radiologist agreements are presented in Table 1. The performance of the analysis controller 110 showed no significant difference with the inter-reader agreement for objective response (OR) prediction (FIGS. 6-7) in the GOYA holdout set (NCT01287741) −0.02 (95% CI: −0.06, 0.02), differences of less than 5% were observed in the other test sets (−0.04, 95% CI: −0.09, −0.01, in GO29365/NCT02257567 and −0.05, 95% CI: −0.07, −0.02, in GO29781/NCT02500407). No statistically significant differences were found for the four-response category accuracy of the analysis controller 110 on the GOYA holdout set (NCT01287741) (−0.04, 95% CI: −0.11, 0.01) and GO29365/NCT02257567 (−0.03, 95% CI: −0.08, 0.03), while a −0.07 (95% CI: −0.11, −0.03) was observed in GO29781/NCT02500407 (FIGS. 6-7 and 8B).

An expert radiologist reviewed the intermediate and final outputs of the analysis controller 110 (including tumor masks at screening and follow-up, the level of metabolic activity (e.g., the maximum uptake value (SUVmax) of the hottest lesion), indicator of new lesions, and the predicted metabolic response) to make an assessment of response on 114 interim or end-of-treatment scans. In 81% (95% CI: 74-88) of the visits, no modifications of the predicted response was needed. The average time for the radiologist's review was 2.02 minutes per visit (range 1-15 minutes). The concordance of the model to the expert radiologist was similar to the agreement of the radiologist to the final expert radiologist committee response. The radiologist agreement (FIGS. 8D-E) with the complete metabolic response (CMR) prediction of the analysis controller 110 was 88% (95% CI: 82-93). The agreement with the objective response prediction was 91% (95% CI: 86-96) and the agreement for 4 response classes prediction was 81% (95% CI: 74-88). No statistically significant differences between the radiologist's agreement with the proposed method and the agreement of the radiologist with the final expert radiologist committee response (FIG. 8E) were observed for the objective response assessments (−0.01, 95% CI: −0.07, 0.05) and the 4 response classes assessments (−0.05, 95% CI: −0.14, 0.4) while a −0.09 (95% CI: −0.16, −0.03) difference was observed for the complete metabolic response (CMR) assessments accuracy.

Survival analysis was performed for the three test datasets. The predicted complete metabolic response (CMR) was strongly prognostic for overall survival (OS) and investigator assessed progression free survival (PFS) (Table 2, FIGS. 7(C) and 8D). The overall survival (OS) hazard ratios (HRs) for patients identified by the analysis controller 110 as exhibiting complete metabolic response (CMR) at end of treatment were 0.123 (95% CI: 0.055, 0.276) in the GOYA holdout set (NCT01287741), 0.205 (95% CI: 0.098, 0.426) in the R/R DLBCL bendamustine+rituximab (BR) and polatuzumab (pola)+BR cohorts in GO29365/NCT02257567 and 0.054 (95% CI: 0.01, 0.442) in the GO29781/NCT02500407 phase 2 R/R FL expansion cohort. The progression free survival (PFR) hazard ratios (HRs) and the hazard ratios of the responses from the expert radiologist committee are shown in Table 2 below.

TABLE 2

| | | |
|---|---|---|
| N | OS HR | PFS HR |

GOYA Holdout (NCT01287741) (R-CHOP or G-CHOP)

| | | | |
|---|---|---|---|
| Model | 179 | 0.123 | 0.199 |
| | | (0.055, 0.276) | (0.105, 0.376) |
| Final IRC | | 0.226 | 0.225 |
| response | | (0.103, 0.496) | (0.124, 0.408) |
| Radiologist 1 | | 0.278 | 0.281 |
| | | (0.123, 0.625) | (0.153, 0.516) |
| Radiologist 2 | | 0.175 | 0.202 |
| | | (0.078, 0.394) | (0.108, 0.377) |

GO29781/NCT02500407 (mosunetuzumab)

| | | | |
|---|---|---|---|
| Model | 58 | 0.054 | 0.162 |
| | | (0.01, 0.442) | (0.074, 0.358) |
| Final IRC | | 0.292 | 0.120 |
| response | | (0.072, 1.175) | (0.053, 0.274) |
| Radiologist 1 | | 0.445 | 0.141 |
| | | (0.119, 1.668) | (0.063, 0.316) |
| Radiologist 2 | | 0.280 | 0.103 |
| | | (0.070, 1.126) | (0.044, 0.239) |

GO29365/NCT02257567 (bendamustine +
rituximab +/− polatuzumab vedotin)

| | | | |
|---|---|---|---|
| Model | 82 | 0.205 | 0.297 |
| | | (0.098, 0.426) | (0.165, 0.535) |
| Final IRC | | 0.272 | 0.157 |
| response | | (0.137, 0.524) | (0.086, 0.285) |
| Radiologist 1 | | 0.287 | 0.255 |
| | | (0.144, 0.570) | (0.145, 0.450) |
| Radiologist 2 | | 0.448 | 0.413 |
| | | (0.222, 0.903) | (0.234, 0.731) |

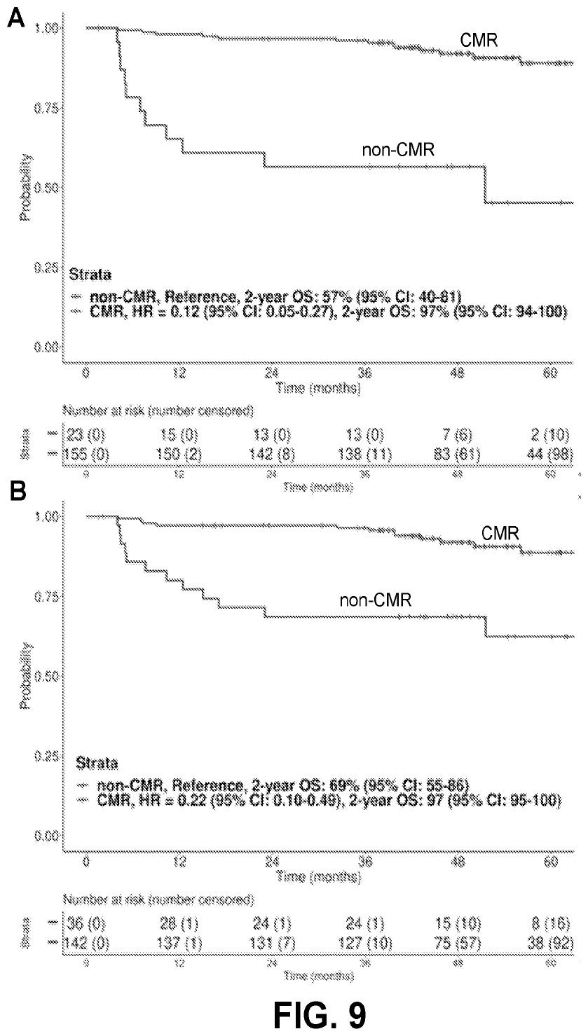

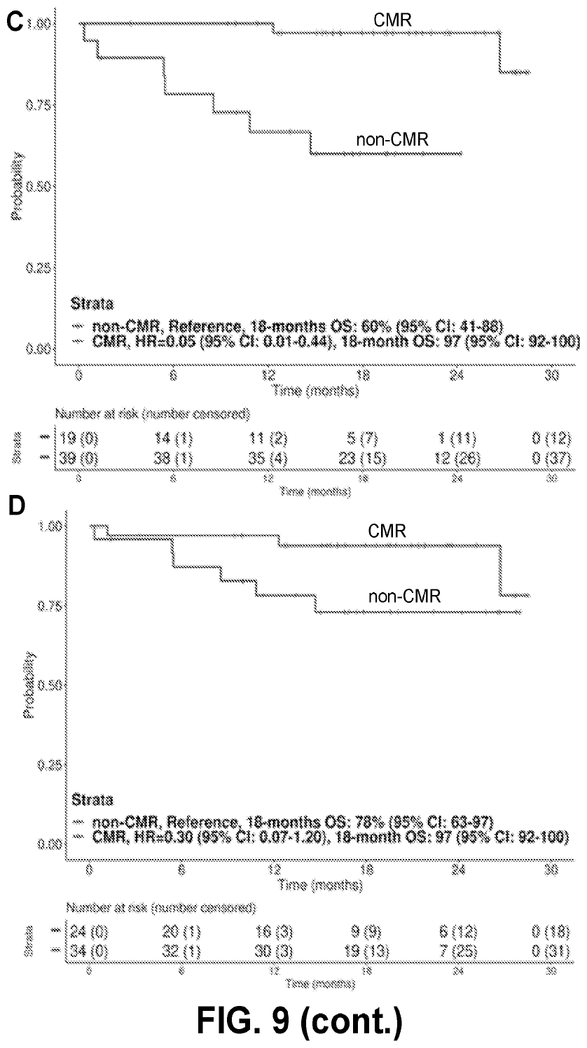

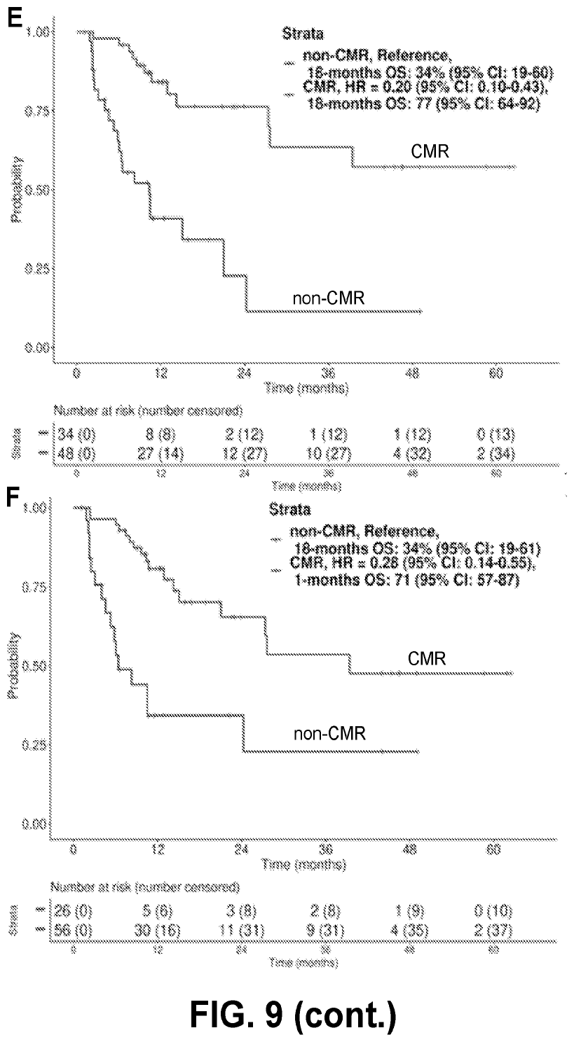

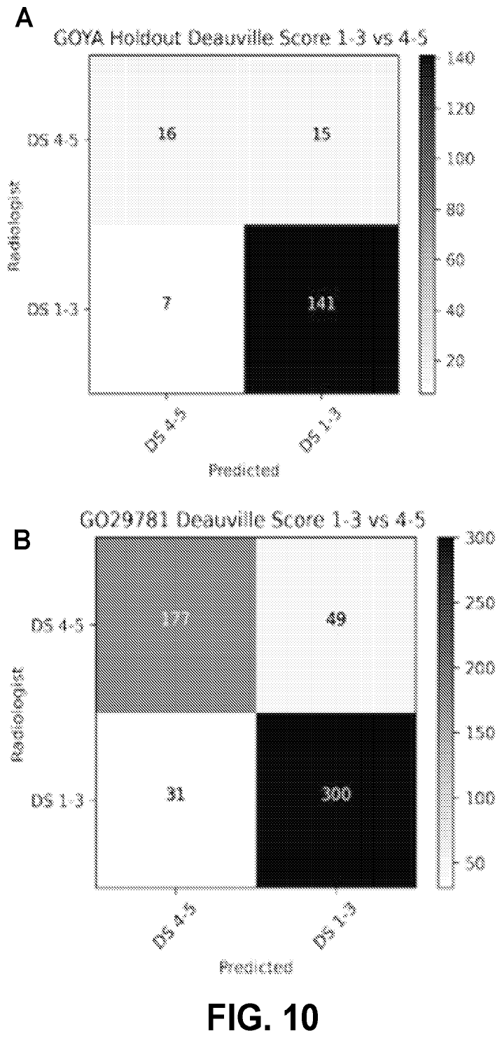

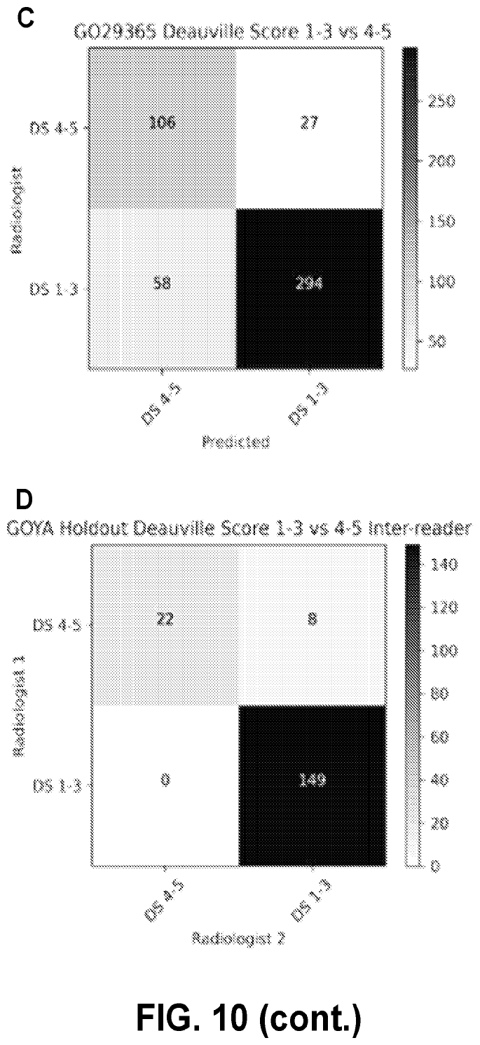

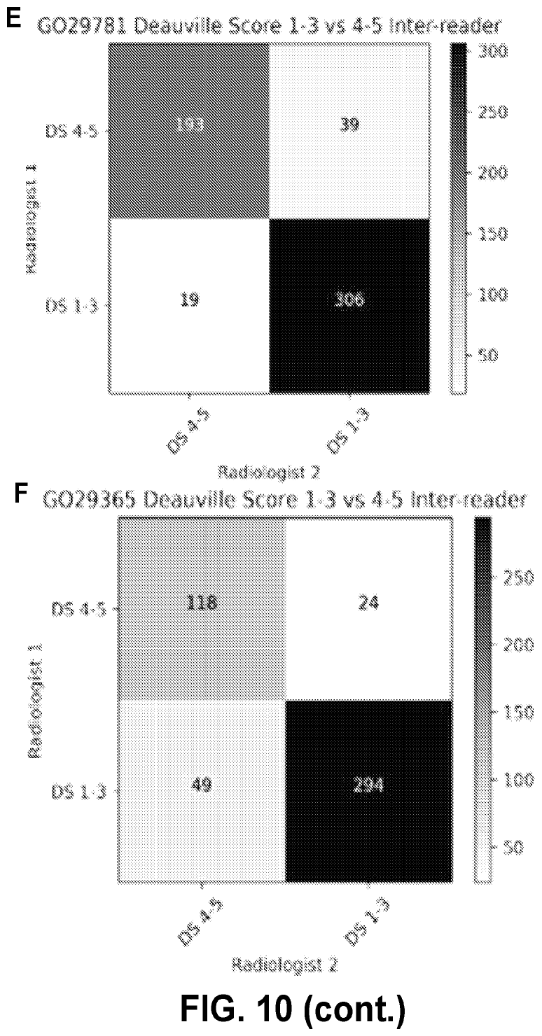

Those patients predicted as exhibiting complete metabolic response by the analysis controller 110 were at higher or equal death risk at landmark survival times compared with the patients identified by the expert radiologist committee as not exhibiting complete metabolic response (CMR) (FIG. 9). The 2-year overall survival for predicted non-complete metabolic response (non-CMR) patients in the GOYA holdout set (NCT01287741) (FIG. 9(A)) is 57% (95% CI: 40-81) compared with 69% (95% CI: 55-86) for non-complete metabolic response (CMR) patients identified by the expert radiologist committee (FIG. 9(D)). The 18-months overall survival (OS) for complete metabolic response patience identified by the analysis controller 110 in the GO29781/NCT02500407 dataset (FIG. 9(B)) and the GO29365/NCT02257567 dataset (FIG. 9(C)) were 60% (95% CI: 41-88) and 34% (95% CI: 19-60), respectively, compared with 78% (95% CI: 63-97, FIG. 9(E)) and 34% (95% CI: 19-61, FIG. 9(F)) for those identified by the expert radiologist committee. Patience identified by the analysis controller 110 as exhibiting complete metabolic response (CMR) had higher or equal survival rates compared with those identified by the expert radiologist committee at the same landmark survival times in the three test sets (FIG. 7). Kaplan-Meier curves and progression free survival (PFS) estimates for progression free survival (PFS) are presented in FIG. 9. Expert radiologist and the analysis controller 110 made assessments of the Deauville score (DS) based on the FDG uptake for all three datasets are shown in FIG. 10.

The log rank tests for overall survival based on CMR versus non-CMR of analysis controller 110 and the final IRC responses for each test set. This analysis demonstrates that there was not a significant difference between the results from the analysis controller 110 and the final IRC response for all but one group (GO29781, Final IRC response) because the p values are less than 0.05 (Table 3).

TABLE 3

| | Response by the AI | Final IRC |
|---|---|---|
| | Model Chi-square | response Chi- |
| | (p-val) | square (p-val) |
| GOYA Holdout | 36.5 | 16.5 |
| | (p < 10⁻⁸) | (p < 10⁻⁴) |
| GO29781 | 14.3 | 3.4 |
| | (p < 10⁻³) | (p = 0.07) |
| GO29365 | 21.6 | 15.6 |
| | (p < 10⁻⁵) | (p < 10⁻⁴) |

Segmentation and lesion detection performance of example longitudinal segmentation models 113 on the follow up FDG-PET/CT scans in the GOYA (NCT01287741) set was conducted. A longitudinal segmentation model 113 is developed to refine tumor segmentation on registered follow-up FDG-Pet/CT scans. First, a region-specific VNet model is trained to refine tumor segmentation for different areas (e.g., abdomen/pelvis area, the chest area, and the head/neck area). The inputs to the VNets consist of a patch of 4 modalities—the screening PET, the screening CT, the registered follow-up PET and CT—centered on the centroid of the tumors predicted at follow-up by the single time point tumor segmentation. Chest and head/neck VNets inputs are 96*96*96*4 while the abdomen/pelvis VNet input size is 128*128*128*4. The predicted patch tumor mask is then inserted into the whole body tumor mask. Unet and Swin UNETR, pre-trained models, were also tested for longitudinal lesion segmentation on the registered FDG-PET/CT follow-up scans. Ablation studies were also performed to assess the performance gained for lesion segmentation on follow-up scans by the addition of the longitudinal segmentation model, and the performance gained for lesion segmentation on follow-up scans by the addition of the screening FDG-PET/CT scan information as an input to the longitudinal segmentation model.

Using the GOYA training set, longitudinal tumor segmentation models were trained on 1919 samples from 740 follow-up scans registered to their baseline scans (including 384 scans without any tumors); 1196 patches were in the abdomen/pelvis area (397 negative examples); 379 in the chest area (54 negative examples) and 344 in the head/neck area (85 negative examples). The GOYA test set comprised 179 registered follow-up scans In the test set, the abdomen/pelvis VNet achieves a Dice score (DSC) of 0.802 while the chest and head/neck VNets achieve dice scores of 0.813 and 0.778, respectively. The overall dice score at the entire scan-level was 0.796. The final tumor masks have an average number of 0.11 false positive (FP) lesions, 0.39 false negative (FN) and 1.47 true positive (TP) lesions per scan (Table 4). The addition of the step of the longitudinal segmentation VNet allows the reduction of false positives (average of 1.21 false positive lesions per scan when using the tumor masks from the single time point tumor segmentation models). The use of both screening and follow-up FDG-PET/CT information in the longitudinal segmentation models allow to increase the sensitivity to lesions in follow-up scans compared to a model using only the follow-up scans as input (average of 0.65 false negative lesions per scan when using only the follow-up scan as input). The VNet showed superior performance compared to UNet and Swin UNETR. for longitudinal segmentation. A detailed assessment of the performance of the different models is presented in Table 4.

TABLE 4

| Model | TP | FP | FN | WB DSC | Abdomen DSC | Chest DSC | Head DSC |
|---|---|---|---|---|---|---|---|
| VNet | 1.47 | 0.11 | 0.39 | 0.796 ± 0.11 | 0.802 | 0.813 | 0.778 |
| VNet_{follow-up only} | 1.22 | 0.09 | 0.65 | 0.741 ± 0.14 | 0.753 | 0.762 | 0.734 |
| UNet | 1.33 | 0.17 | 0.52 | 0.780 ± 0.13 | 0.785 | 0.798 | 0.713 |
| Swin UNETR | 1.42 | 0.13 | 0.43 | 0.787 ± 0.11 | 0.791 | 0.805 | 0.781 |
| No longitudinal | 1.51 | 1.21 | 0.34 | 0.623 ± 0.16 | 0.607 | 0.749 | 0.6 |
| segmentation | | | | | | | |

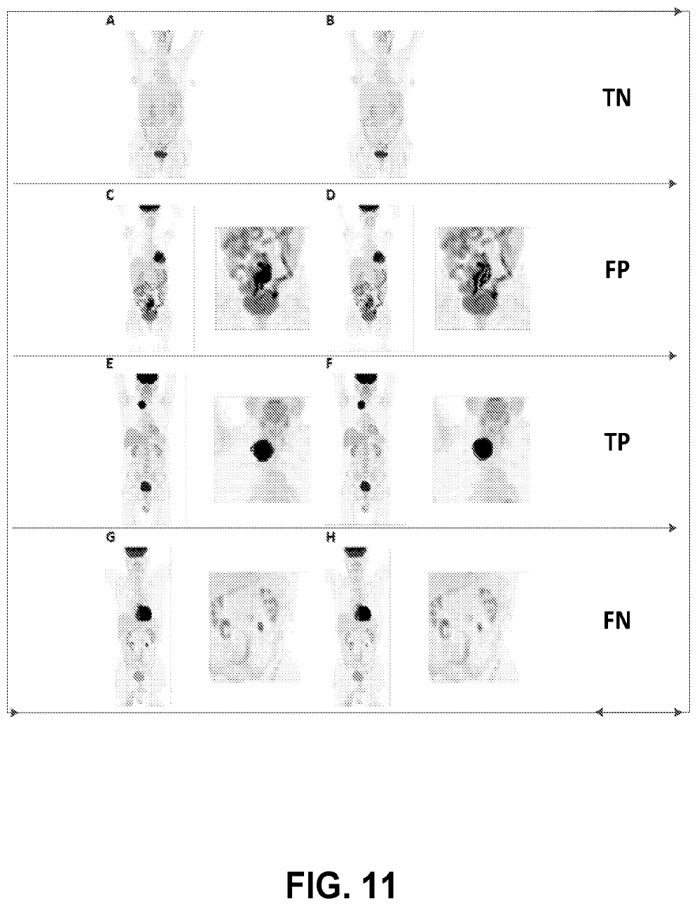

FIG. 11 depicts examples of true positives (TP), true negatives (TN), false positives (FP), and false negatives (FN). FIGS. 11A and 11B demonstrate a true negative example of correctly classified CMR with the A) absence of tumors in the ground truth TMTV annotations and B) absence of tumors in the method's tumor segmentation. FIGS. 11C and 11D demonstrate a false positive example of CMR with C) no metabolically active tumors according to the IRC annotations and D) a False positive lesion from the method. FIGS. 11E and 11F demonstrate a true positive example correctly classified non-CMR with the same lesion detected by E) the IRC and F) the model. FIGS. 11G and 11H demonstrate a false negative example of PMD G) by the IRC with a small lesion misassessed as CMR H) by the model.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:

Item 1: A computer-implemented method, comprising: determining, based at least on a first positron emission tomography (PET) scan and a first computed tomography (CT) scan from a first timepoint, a first tumor mask corresponding to a first lesion present in the first PET scan and the first CT scan; determining, based at least on a second PET scan and a second CT scan from a second timepoint, a second tumor mask corresponding to a second lesion present in the second PET scan and the second CT scan; applying a longitudinal segmentation model to update, based at least on the first PET scan, the first CT scan, the second PET scan, and the second CT scan, each of the first tumor mask and the second tumor mask; and determining, based on at least one of the first updated tumor mask and the second updated tumor mask, a response to a treatment for a disease.

Item 2: The method of Item 1, wherein the first tumor mask identifies a first plurality of pixels in each of the first PET scan and the first CT scan depicting the first lesion, and wherein the second tumor mask identifies a plurality of pixels from the second PET scan and the second CT scan depicting the second lesion.

Item 3: The method of any of Items 1 to 2, further comprising: registering the first CT scan, the first PET scan, the second CT scan, and the second PET scan in order to align the first CT scan and the first PET scan with the second CT scan and the second PET scan.

Item 4: The method of any one of Items 1 to 3, further comprising: identifying, based at least on the first updated tumor mask and the second updated tumor mask, the second lesion as a new lesion; and in response to the second lesion being identified as the new lesion, determining the response to the treatment as progressive disease (PMD).

Item 5: The method of Item 4, further comprising: determining, based at least on the first updated tumor mask and the second updated tumor mask, a distance between the first lesion and the second lesion; identifying the second lesion as the new lesion based at least on the distance between the first lesion and the second lesion satisfying one or more thresholds; and identifying the second lesion as a same lesion as the first lesion based at least on the distance between the first lesion and the second lesion failing to satisfy the one or more thresholds.

Item 6: The method of any one of Items 4 to 5, further comprising: in response to determining that the first lesion and the second lesion are a same lesion, determining the response to the treatment for the disease based at least on a change in metabolic activity exhibited by the lesion between the first timepoint and the second timepoint.

Item 7: The method of Item 6, wherein the change in metabolic activity between the first timepoint and the second timepoint is determined by at least determining, based at least on the first updated tumor mask and the first PET scan, a first level of metabolic activity exhibited by the lesion at the first timepoint, determining, based at least on the second updated tumor mask and the second PET scan, a second level of metabolic activity exhibited by the lesion at the second timepoint, and determining, based at least on the first level of metabolic activity and the second level of metabolic activity, the change in metabolic activity between the first timepoint and the second timepoint.

Item 8: The method of Item 7, wherein the response to the treatment is determined as progressive metabolic disease (PMD) based at least on the change in metabolic activity between the first timepoint and the second timepoint satisfying a first threshold.

Item 9: The method of Item 8, wherein the response to the treatment is determined as no metabolic response (NMR) based at least on the change in metabolic activity between the first timepoint and the second timepoint satisfying a second threshold but failing to satisfy the first threshold.

Item 10: The method of Item 9, wherein the response to the treatment is determined as partial metabolic response (PMR) based at least on the change in metabolic activity between the first timepoint and the second timepoint failing to satisfy the first threshold and the second threshold.

Item 11: The method of any one of Item 7 to 10, wherein the first level of metabolic activity corresponds to a first standardized uptake value (SUV) and the second level of metabolic activity corresponds to a second standardized uptake (SUV) value.

Item 12: The method of any one of Item 7 to 10, wherein each of the first level of metabolic activity and the second level of metabolic activity correspond to a maximum, a minimum, a median, a mean, or a mode level of metabolic activity exhibited by the lesion at a corresponding timepoint.

Item 13: The method of any one of Items 1 to 12, wherein the first CT scan and the first PET scan are performed prior to the treatment for the disease, and wherein the second CT scan and the second PET scan are performed subsequent to the treatment for the disease.

Item 14: The method of any one of Items 1 to 13, further comprising: determining, based at least on the first updated tumor mask and the second updated tumor mask, a change in tumor volume; and determining, based at least on the change in tumor volume, the response to the treatment for the disease.

Item 15: The method of any one of Items 1 to 13, further comprising: determining, based at least on the first updated tumor mask and the second updated tumor mask, a variance in a first change in metabolic activity and/or a second change in tumor volume between the first timepoint and the second timepoint across different lesions; and determining the response to the treatment based at least on the variance in the change in metabolic activity and/or tumor volume between the first timepoint and the second timepoint exhibited by the different lesions.

Item 16: The method of any one of Items 1 to 15, further comprising: determining, based at least on the first updated tumor mask and the second updated tumor mask, a progression of the disease.

Item 17: The method of any one of Items 1 to 16, wherein the first tumor mask is determined by applying a segmentation model to the first PET scan and the first CT scan, and wherein the second tumor mask is determined by applying the segmentation model to the second PET scan and the second CT scan.

Item 18: The method of any one of Items 1 to 17, wherein the longitudinal segmentation model is an artificial neural network or a vision transformer.

Item 19: The method of any one of Items 1 to 18, wherein each of the first CT scan, the first PET scan, the second CT scan, and the second PET scan is a three-dimensional volume comprising a plurality of two-dimensional patches.

Item 20: The method of any one of Items 1 to 19, wherein each pixel in the first PET scan and the second PET scan is associated with an intensity value corresponding to a level of metabolic activity.

Item 21: The method of any one of Items 1 to 20, wherein each pixel in the first CT scan and the second CT scan is associated with an intensity value corresponding to a tissue density or X-ray attenuation.

Item 22: The method of any one of Items 1 to 21, further comprising: training the longitudinal segmentation model to update two or more tumor masks, each tumor mask of the two or more tumor masks being generated from a positron emission tomography (PET) scan and a computed tomography (CT) scan from a single timepoint.

Item 23: The method of any one of Items 1 to 22, wherein the response to the treatment for the disease is complete metabolic response (CMR) or non-complete metabolic response (non-CMR).

Item 24: The method of any one of Items 1 to 22, wherein the response to the treatment for the disease is responder or non-responder.

Item 25: The method of one any one of Items 1 to 22, wherein the response to the treatment for the disease is complete metabolic response (CMR), partial metabolic response (PMR), no metabolic response (NMR), or progressive metabolic disease (PMD).

Item 26: The method of any one of Items 1 to 25, further comprising: extracting, from the first PET scan and the first CT scan, a first patch including the first lesion associated with first tumor mask; extracting, from the second PET scan and the first CT scan, a second patch including the second lesion associated with the second tumor mask; and applying the longitudinal segmentation model to the first patch and the second patch in order to update each of the first tumor mask and the second tumor mask.

Item 27: A system, comprising: at least one data processor; and at least one memory storing instructions, which when executed by the at least one data processor, result in operations comprising the method of any of Items 1 to 26.

Item 28: A non-transitory computer readable medium storing instructions, which when executed by at least one data processor, result in operations comprising the method of any of Items 1 to 26.

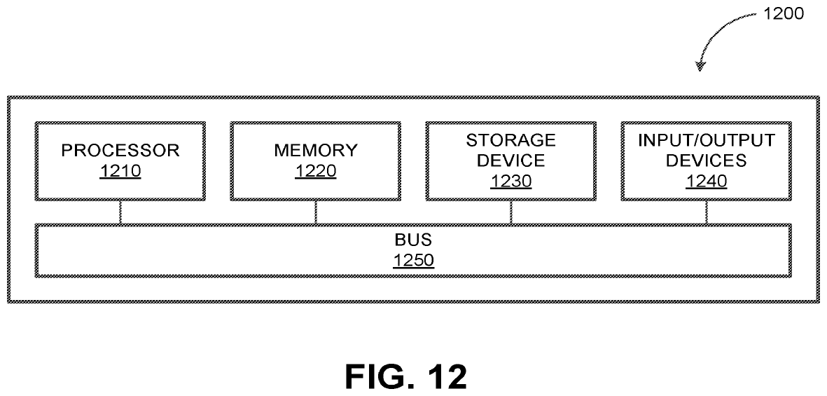

FIG. 12 depicts a block diagram illustrating an example of a computing system 1200 consistent with implementations of the current subject matter. Referring to FIGS. 1-10, the computing system 1200 can be used to implement the analysis controller 110, the one or more imaging devices 120, the client device 130, and/or any components therein.

As shown in FIG. 12, the computing system 1200 can include a processor 1210, a memory 1220, a storage device 1230, and an input/output device 1240. The processor 1210, the memory 1220, the storage device 1230, and the input/output device 1240 can be interconnected via a system bus 1250. The processor 1210 is capable of processing instructions for execution within the computing system 1200. Such executed instructions can implement one or more components of, for example, the analysis controller 110, the one or more imaging devices 120, and the client device 130. In some example embodiments, the processor 1210 can be a single-threaded processor. Alternately, the processor 1210 can be a multi-threaded processor. The processor 1210 is capable of processing instructions stored in the memory 1220 and/or on the storage device 1230 to display graphical information for a user interface provided via the input/output device 1240.

The memory 1220 is a computer readable medium such as volatile or non-volatile that stores information within the computing system 1200. The memory 1220 can store data structures representing configuration object databases, for example. The storage device 1230 is capable of providing persistent storage for the computing system 1200. The storage device 1230 can be a solid state drive, a floppy disk device, a hard disk device, an optical disk device, or a tape device, or other suitable persistent storage means. The input/output device 1240 provides input/output operations for the computing system 1200. In some example embodiments, the input/output device 1240 includes a keyboard and/or pointing device. In various implementations, the input/output device 1240 includes a display unit for displaying graphical user interfaces.

According to some example embodiments, the input/output device 1240 can provide input/output operations for a network device. For example, the input/output device 1240 can include Ethernet ports or other networking ports to communicate with one or more wired and/or wireless networks (e.g., a local area network (LAN), a wide area network (WAN), the Internet).

In some example embodiments, the computing system 1200 can be used to execute various interactive computer software applications that can be used for organization, analysis and/or storage of data in various formats. Alternatively, the computing system 1200 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1240. The user interface can be generated and presented to a user by the computing system 1200 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random query memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, recurrent provided to the user can be any form of sensory recurrent, such as for example visual recurrent, auditory recurrent, or tactile recurrent; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
determining, based at least on a first positron emission tomography (PET) scan and a first computed tomography (CT) scan from a first timepoint, a first tumor mask corresponding to a first lesion present in the first PET scan and the first CT scan;
determining, based at least on a second PET scan and a second CT scan from a second timepoint, a second tumor mask corresponding to a second lesion present in the second PET scan and the second CT scan;
applying a longitudinal segmentation model to update, based at least on the first PET scan, the first CT scan, the second PET scan, and the second CT scan, each of the first tumor mask and the second tumor mask; and
determining, based on at least one of the first updated tumor mask and the second updated tumor mask, a response to a treatment for a disease.

2. The system of claim 1, wherein the first tumor mask identifies a first plurality of pixels in each of the first PET scan and the first CT scan depicting the first lesion, and wherein the second tumor mask identifies a plurality of pixels from the second PET scan and the second CT scan depicting the second lesion.

3. The system of claim 1, further comprising:
registering the first CT scan, the first PET scan, the second CT scan, and the second PET scan in order to align the first CT scan and the first PET scan with the second CT scan and the second PET scan.

4. The system of claim 1, further comprising:
identifying, based at least on the first updated tumor mask and the second updated tumor mask, the second lesion as a new lesion;
in response to the second lesion being identified as the new lesion, determining the response to the treatment as progressive disease (PMD);
determining, based at least on the first updated tumor mask and the second updated tumor mask, a distance between the first lesion and the second lesion;
identifying the second lesion as the new lesion based at least on the distance between the first lesion and the second lesion satisfying one or more thresholds; and
identifying the second lesion as a same lesion as the first lesion based at least on the distance between the first lesion and the second lesion failing to satisfy the one or more thresholds.

5. (canceled)

6. The system of claim 4, further comprising:
in response to determining that the first lesion and the second lesion are a same lesion,
determining a change in metabolic activity exhibited by the lesion between the first time point and the second timepoint by at least
determining, based at least on the first updated tumor mask and the first PET scan, a first level of metabolic activity exhibited by the lesion at the first timepoint,
determining, based at least on the second updated tumor mask and the second PET scan, a second level of metabolic activity exhibited by the lesion at the second timepoint, and
determining, based at least on the first level of metabolic activity and the second level of metabolic activity, the change in metabolic activity between the first timepoint and the second timepoint,
determining the response to the treatment for the disease based at least on the change in metabolic activity exhibited by the lesion between the first timepoint and the second timepoint.

7. (canceled)

8. The system of claim 6, wherein the response to the treatment is determined to be
(i) progressive metabolic disease (PMD) based at least on the change in metabolic activity between the first timepoint and the second timepoint satisfying a first threshold,
(ii) no metabolic response (NMR) based at least on the change in metabolic activity between the first timepoint and the second timepoint satisfying a second threshold but failing to satisfy the first threshold, or
(iii) partial metabolic response (PMR) based at least on the change in metabolic activity between the first timepoint and the second timepoint failing to satisfy the first threshold and the second threshold.

9. (canceled)

10. (canceled)

11. The system of claim 6, wherein the first level of metabolic activity corresponds to a first standardized uptake value (SUV) and the second level of metabolic activity corresponds to a second standardized uptake (SUV) value.

12. The system of claim 6, wherein each of the first level of metabolic activity and the second level of metabolic activity correspond to a maximum, a minimum, a median, a mean, or a mode level of metabolic activity exhibited by the lesion at a corresponding timepoint.

13. The system of claim 1, wherein the first CT scan and the first PET scan are performed prior to the treatment for the disease, and wherein the second CT scan and the second PET scan are performed subsequent to the treatment for the disease.

14. The system of claim 1, further comprising:
determining, based at least on the first updated tumor mask and the second updated tumor mask, a change in tumor volume; and
determining, based at least on the change in tumor volume, the response to the treatment for the disease.

15. The system of claim 1, further comprising:
determining, based at least on the first updated tumor mask and the second updated tumor mask, a variance in a first change in metabolic activity and/or a second change in tumor volume between the first timepoint and the second timepoint across different lesions; and
determining the response to the treatment based at least on the variance in the change in metabolic activity and/or tumor volume between the first timepoint and the second timepoint exhibited by the different lesions.

16. The system of claim 1, further comprising:
determining, based at least on the first updated tumor mask and the second updated tumor mask, a progression of the disease.

17. The system of claim 1, wherein the first tumor mask is determined by applying a segmentation model to the first PET scan and the first CT scan, and wherein the second tumor mask is determined by applying the segmentation model to the second PET scan and the second CT scan.

18. (canceled)

19. (canceled)

20. The system of claim 1, wherein each pixel in the first PET scan and the second PET scan is associated with an intensity value corresponding to a level of metabolic activity, and wherein each pixel in the first CT scan and the second CT scan is associated with an intensity value corresponding to a tissue density or X-ray attenuation.

21. (canceled)

22. The system of claim 1, further comprising:
training the longitudinal segmentation model to update two or more tumor masks, each tumor mask of the two or more tumor masks being generated from a positron emission tomography (PET) scan and a computed tomography (CT) scan from a single timepoint.

23. The system of claim 1, wherein the response to the treatment for the disease is complete metabolic response (CMR) or non-complete metabolic response (non-CMR).

24. (canceled)

25. The system of claim 1, wherein the response to the treatment for the disease is complete metabolic response (CMR), partial metabolic response (PMR), no metabolic response (NMR), or progressive metabolic disease (PMD).

26. The system of claim 1, further comprising:
extracting, from the first PET scan and the first CT scan, a first patch including the first lesion associated with first tumor mask;
extracting, from the second PET scan and the first CT scan, a second patch including the second lesion associated with the second tumor mask; and
applying the longitudinal segmentation model to the first patch and the second patch in order to update each of the first tumor mask and the second tumor mask.

27. A computer implemented method, comprising:
determining, based at least on a first positron emission tomography (PET) scan and a first computed tomography (CT) scan from a first timepoint, a first tumor mask corresponding to a first lesion present in the first PET scan and the first CT scan;
determining, based at least on a second PET scan and a second CT scan from a second timepoint, a second tumor mask corresponding to a second lesion present in the second PET scan and the second CT scan;
applying a longitudinal segmentation model to update, based at least on the first PET scan, the first CT scan, the second PET scan, and the second CT scan, each of the first tumor mask and the second tumor mask; and
determining, based on at least one of the first updated tumor mask and the second updated tumor mask, a response to a treatment for a disease.

28. A non-transitory computer readable medium storing instructions, which when executed by at least one data processor, result in operations comprising:
determining, based at least on a first positron emission tomography (PET) scan and a first computed tomography (CT) scan from a first timepoint, a first tumor mask corresponding to a first lesion present in the first PET scan and the first CT scan;
determining, based at least on a second PET scan and a second CT scan from a second timepoint, a second tumor mask corresponding to a second lesion present in the second PET scan and the second CT scan;
applying a longitudinal segmentation model to update, based at least on the first PET scan, the first CT scan, the second PET scan, and the second CT scan, each of the first tumor mask and the second tumor mask; and
determining, based on at least one of the first updated tumor mask and the second updated tumor mask, a response to a treatment for a disease.
